(19) United States
(12) Patent Application Publication
(10) Pub. No.: US 2006/0211002 A1
(43) Pub. Date: Sep. 21, 2006

(54) Antibiotic susceptibility and virulence factor detection in Pseudomonas aeruginosa

(76) Inventors: Jan Weile, Leinfelden-Echterdingen (DE); Milorad Susa, Stuttgart (DE); Rolf Schmid, Stuttgart (DE); Till T. Bachmann, Stuttgart (DE); Cornelius Knabbe, Stuttgart (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/084,700

(22) Filed: Mar. 18, 2005

(51) Int. Cl. C12Q 1/68; C12M 1/34

(52) U.S. Cl. 435/6; 435/287.2

(57) ABSTRACT

The present invention relates in general to the detection of antibiotic resistance determinants in Pseudomonas aeruginosa (P. aeruginosa). The present invention discloses a micro-array for the detection of antibiotic resistance determinants and mutations in said organism, a method for the detection of said determinants and a kit. This micro-array concept offers the rapid sensitive and specific identification of antibiotic resistance profiles.

### FIELD OF THE INVENTION

The present invention relates in general to the detection of Pseudomonas aeruginosa (P. aeruginosa) strains exhibiting multi-resistance to antibiotics. In particular, the present invention pertains to a micro-array for the detection of antibiotic resistance determinants in said organism, a method for the detection of said determinants and a kit. This micro-array concept offers the rapid, sensitive and specific identification of antibiotic resistance profiles. It is easily expandable and may thus be adapted to changed clinical and epidemiological requirements in clinical diagnosis as well as in epidemiological studies.

### BACKGROUND OF THE INVENTION

P. aeruginosa is an opportunistic pathogen associated with nosocomial infections of immuno-compromised patients especially in intensive care units (ICUs). P. aeruginosa is responsible for approximately 10% of all infections on ICUs and results in a high mortality and morbidity when associated with pneumonia or septicemia (Prevention, C.f.D.C.a.; Am. J. Infect. Control. 24 (1996), 380-388). This organism is characterized by an intrinsic resistance to various antimicrobial agents and an ability to develop multiresistance during antibiotic therapy (Livermore, D. M.; Clinical Infectious Diseases 34 (2002), 634-40). The intrinsic multiresistance results from the synergy between broadly specific drug efflux pumps and a low degree of outer membrane permeability. A variety of efflux systems have been identified to date, including the well characterized MexAB-OprM, MexCD-OprJ, MexEF-OprN and MexXY-OprM efflux pumps (Masuda, N., et al.; Antimicrob. Agents Chemother. 44(12) (2000), 3322-7). MexAB-OprM is constitutively expressed in wild type P. aeruginosa PAO1, whereas the other efflux systems are not. Mutations in regulatory genes of these efflux systems (mexR, mexT, nfxB) can either cause overexpression of MexAB-OprM or may induce expression of the other regulated efflux systems. P. aeruginosa may as well harbour different plasmid encoded antibiotic resistance genes like −lactamases (tem, shv, oxa), aminoglycoside modifying enzymes (aac, aad, aph) and carbapenemases (imp, vim). These plasmids can be easily acquired via horizontal gene transfer from other gram-negative organisms, especially in a clinical setting.

So far, detection of P. aeruginosa has been performed by isolating nucleic acid sequences from clinical samples and analyzing them by either using gel electrophoresis of DNA fragments (e.g. of restriction fragments)—the so-called southern blot, hybridization events, and the direct sequencing of DNA (for example according to the Maxam-Gilbert method). All of the above-mentioned methods are widely used in biological sciences, medicine and agriculture. The deficiencies of the three methods reside, however, in that even though southern blots and hybridization experiments may be carried out relatively fast, they are only useful for the analysis of short DNA strands. The DNA sequencing results in the accurate determination of the nucleic acid sequences, but is time consuming, expensive and connected with certain efforts when applied to greater projects, e.g. the sequencing of a complete genome.

Known methods to detect the presence of P. aeruginosa in a clinical sample reside e.g. in real-time polymerase chain reactions (cf. US 2004/248148) or other PCR based assays (cf. US 2003/180733), which use primers specific for particular genes of said organism. Also, the use of enzyme linked immunosorbant assays and Western blot immunoassays for the detection of P. aeruginosa is disclosed for example in U.S. Pat. No. 6,551,795 and EP 0 265 672.

Since these phenotypic based microbiological and biochemical techniques for species identification and antibiotic susceptibility determination require at least two days, a reliable therapy is not possible in urgent cases of critical ill patients. The development of new and faster methods is therefore a crucial point to allow a better adjustment of the antibiotic treatment of severe infections caused by multiresistant pathogens.

The micro-array technology represents in contrast to e.g. PCR and antibody basing methods, a tool for a highly specific, parallel detection of thousands of different DNA sequences in a single experiment (Schena, M. et al.; Science 270 (1995), 467-470). Micro-arrays which are in some cases also referred to as hybridization arrays, gene arrays or gene chips comprise in brief a carrier or support on which at defined locations at a possibly high density capture molecules are attached directly or via a suitable spacer molecule. The spacer molecules may be considered to function as a “bridge” between the capture molecule and the surface of the carrier to allow an easier attachment of the capture molecule. Said capture molecules consist of relatively short nucleic acid sequences, in particular DNA, which is capable to hybridize specific to the target molecules or probe molecules to be analyzed resulting usually in DNA:DNA or DNA:RNA hybrids. The occurrence of the hybridization event is than detected with for example fluorescent dyes and analyzed.

The advantages of the micro-array concept resides preliminary in its ability to carry out very large numbers of hybridization-based analyses simultaneously. Originally developed for the analysis of mammalian gene expression, an increasing number of reports on micro-arrays for identification and characterization of prokaryotes also used in microbial diagnostics was encountered in recent years (Bodrossy, L. and A. Sessitsch; Curr. Opin. Microbiol. 7 (2004), 245-254). Combination of PCR based pre-amplification steps with subsequent micro-array based detection of amplicons on a micro-array facilitates the sensitive and highly specific detection of PCR products (Call, D. R. et al.; Int. J. Food Microbiol. 67 (2001), 71-80). Amplicons are identified by a specific hybridization reaction on the array thus reducing the risk of wrong positive results due to the occurrence of nonspecific bands after PCR. Besides that, micro-arrays utilizing oligonucleotides as capture probes enable the detection of single nucleotide polymorphisms (SNPs) such as resistance mutations without the need for additional sequencing. However, only a few studies describe the development of diagnostic micro-arrays for the molecular detection of bacterial antibiotic resistance, targeting either a limited number of acquired antibiotic resistance genes or resistance mutations in various genes.

The use of micro-arrays for the detection of pathogenic bacteria is for example disclosed in WO 03/031654, wherein a micro-array with probes for genotyping Mycobacteria species, differentiating Mycobacterium strains and detecting antibiotic-resistant strains is specified. The simultaneous performance on multiple clinical isolates through a single test of a Mycobacterium genotyping test, M. tuberculosis strain differentiation test and an antibiotic-resistance detection test is specified.

WO 01/7737 relates to the identification (detection and/or quantification) of (micro-) organisms among others having homologous nucleotide sequences by identification of their nucleotide sequences, after amplification by a single primer pair. Organisms of the same genus or family may and/or related genes in a specific (micro) organism present in a biological sample may be identified or quantified.

Methods for assaying drug resistance and kits for performing such assays are disclosed in the U.S. Pat. No. 6,013,435. Target sequences associated with genetic elements are selectively amplified and detected. The methods described herein are especially useful for screening of Microorganisms, which are difficult to culture.

In US 2003143591 methods and strategies to detect and/or quantify nucleic acid analytes in micro-array applications such as genotyping (SNP analysis) are disclosed. Nucleic acid probes with covalently conjugated dyes are attached either to adjacent nucleotides or at the same nucleotide of the probe while novel linker molecules attach the dyes to the probes.

The disadvantages of the techniques according to the state of the art for the detection of P. aeruginosa reside in that they require long runs and are solely adaptive to a limited number of samples to be tested and often also expensive. Additionally, no method is known which uses simultaneously several nucleic acids probe for the detection of multiple antibiotic resistance determinants and optionally other virulence factors to facilitate an overview on the resistance properties of a single strain and gives valuable and sometimes life-saving information about a suitable treatment.

### SUMMARY OF THE INVENTION

The present invention provides a micro-array as a genotype based method for detecting antibiotic susceptibility of P. aeruginosa, which incorporates nucleic acids for targeting determinants of multi-resistant P. aeruginosa and optionally specific controls. The micro-array enables a rapid, accurate and inexpensive identification of antibiotic resistance profiles of P. aeruginosa. The inclusion of nucleic acids representing virulence factors, like toxins or alginate, broadens the information about the virulence potential of P. aeruginosa at the same time. Said micro-array is easily expandable and may thus be adapted to changing clinical and epidemiological requirements in clinical diagnosis as well as in epidemiological studies. A fast and reliable assay with a high throughput may be helpful in reducing the spread of multiresistant isolates and improves the treatment options of severe and often life-threatening P. aeruginosa infections.

### BRIEF DESCRIPTION OF THE DRAWINGS

In FIG. 1, an embodiment of a micro-array according to the invention is shown. All capture probes were spotted in triplicates. The mutation position is assigned for single nucleotide polymorphisms (SNPs) and the insertions and deletions of respective genes. Modifying enzyme genes are named according to their substrate specificity. Genes relevant for resistance by their presence were named with the usual name. The different genes are indicated in the array legend. For SNPs, the central base in the probe A, T, G, C is spotted in one row below the other, for insertions and deletions, a wild-type probe below a mutation probe, and for gene presence an anti-sense down to sense probe.

FIG. 2 shows a genotype analysis of respective resistance and virulence genes of the clinical P. aeruginosa isolate No. 23 (b), which was performed using the inventive micro-array and were compared with wild-type P. aeruginosa PAO1 (a). The signal intensity is shown in false color, in intensity increasing from grey to white. The frames highlight the positions in which the two isolates differ from each other.

In FIG. 3, the percent of mismatch probes depending on the mismatch positions (MM)/perfect match position (PM) ratio from all hybridization experiments of the P. aeruginosa test collective is shown.

FIG. 4 shows a genotype analysis by the present micro-array of 3 consecutive P. aeruginosa isolates collected from the same patient Array detail of the 3 isolates (No. 1=a, No. 2=b, No. 3=c) covering gyrA and parC and aminoglycoside modifying enzymes is shown. The signal intensity is shown in false color, in increasing intensity from grey to white.

### DETAILED DESCRIPTION OF THE PREFERRED EMBODIMENT

### Definitions

The term “micro-array” as used herein refers to a carrier or support respectively, which is preferably solid and has a plurality of molecules bound to its surface at defined locations or localized areas. The molecules bound to the carrier comprise nucleic acid sequences, the capture molecules, which are specific for a given or desired target sequence. The sequences may be bound to the carrier via spacer molecules, which bind each capture nucleotide to the surface of the support. In the above context a localized area is an area of the carrier's surface, which contains capture molecules, preferably attached by means of spacers to the surface of the carrier, and which capture molecules are specific for a determined target/probe molecule.

“Spacers” are molecules that are characterized in that they have a first end attached to the biological material and a second end attached to the solid carrier. Thus, the spacer molecule separates the solid carrier and the biological material, but is attached to both. The spacers may be synthesized directly on or may be attached as a whole to the solid carrier at the specific locations, whereby masks may be used at each step of the process. The synthesis comprises the addition of a new nucleotide on an elongating nucleic acid in order to obtain a desired sequence at a desired location by for example photolithographic technologies which are well known to the skilled person. Bindings within the spacer may include carbon-carbon single bonds, carbon-carbon double bonds, carbon-nitrogen single bonds, or carbon-oxygen single bonds. The spacer may be also designed to minimize template independent noise, which is the result of signal detection independent (in the absence) of the template. In addition, the spacer may have side chains or other substitutions. The active group may be reacted by suitable means to form for example preferably a covalent bound between the spacer and solid carrier, capture or probe molecule. Suitable means comprise for example light. The reactive group may be optionally masked/protected initially by protecting groups. Among a wide variety of protecting groups, which are useful are for example FMOC, BOC, t-butyl esters, t-butyl ethers. The reactive group is used to build to attach specifically thereto (after the cleavage of the protecting group) another molecule.

The “localized area” is either known/defined by the construction of the micro-array or is defined during or after the detection and results in a specific pattern. A spot is the area where specific target molecules are fixed on their capture molecules and approved by a detector.

As used herein, the term “carrier” or “support” refers to any material that provides a solid or semi-solid structure and a surface allowing attachment of molecules. Such materials are preferably solid and include for example metal, glass, plastic, silicon, and ceramics as well as textured and porous materials. They may also include soft materials for example gels, rubbers, polymers, and other non-rigid materials. Preferred solid carriers are nylon membranes, epoxy-glass and borofluorate-glass. Solid carriers need not be flat and may include any type of shape including spherical shapes (e.g., beads or microspheres). Preferably solid carriers have a flat surface as for example in slides (such as object slides) and micro-titer plates, wherein a micro-titre plate is a dished container having at least two wells.

The expression “attached” describes a non-random chemical or physical interaction by which a connection between two molecules is obtained. The attachment may be obtained by means of a covalent bond. However, the attachments need not be covalent or permanent. Other kinds of attachment include for example the formation of metalorganic and ionic bonds, binding based on van der Waal's forces, or any kind of enzyme substrate interactions or the so called affinity binding. An attachment to the surface of a carrier or carrier may be also referred to as immobilization.

A “determinant” relates to a factor responsible for the development of resistance in P. aeruginosa, which may be acquired by the micro-organism via horizontal gene transfer and which actively counteracts the effect of an antibiotic. Particularly, the genes conveying resistance to antibiotics, such as mexR, mexT, nfxB, mucA, parC, gyrA, exoU, exoS, exoT, pse, oxa, imp, vim, aac, aph and aad, which may be normally present on plasmid(s) or also may be incorporated in the genome of P. aeruginosa, are envisaged. Also virulent factors, such as e.g. genes involved in the synthesis of toxins and alginate are comprised by said term.

The terms “complementary” or “complementarity” are used in reference to polynucleotides (i.e., a sequence of nucleotides such as an oligonucleotide or a target nucleic acid) in the light of the base-pairing rules. Complementarity may be partial, in which only some bases of the nucleic acids are matched according to the base pairing rules. Alternatively, there may be a complete complementarity between the nucleic acids in such a way that there are no mismatches. The degree of complementarity between nucleic acid strands has significant effects on the stringency and strength of the hybridization between two different nucleic acid strands. Complementarity as used herein is not limited to the predominant natural base pairs. Rather, the term also encompasses alternative, modified and non-natural bases, including but not limited to those that pair with modified or alternative patterns of hydrogen. With regard to complementarity, it is important for some applications to determine whether the hybridization represents a complete or partial complementarity. If it is desired for example to detect the presence or absence of a particular DNA (such as from a virus, bacterium, fungi or protozoan), the only important condition is that the hybridization method ensures hybridization when the relevant sequence is present. Other applications in contrast, may require that the hybridization method distinguish between partial and complete complementarity, for example in the detection of genetic polymorphisms.

The term “homology” and “homologous” refers to a degree of identity. There may be partial homology or complete homology. A partially homologous sequence is one that is less than 100% identical to another sequence.

“Hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, and the melting temperature of the formed hybrid. Hybridization involves the annealing of one nucleic acid to another complementary nucleic acid, i.e., a nucleic acid having a complementary nucleotide sequence.

“Stringency” refers to the conditions, which are involved in a correct hybridization event, for example temperature, ionic strength, pH and the presence of other compounds, under which nucleic acid hybridizations are conducted. Under conditions of high stringency, nucleic acid base pairing will occur only between nucleic acid fragments that have a high frequency of complementary base sequences. Thus, conditions of weak or low stringency are often required when it is desired that nucleic acids that are not completely complementary to one another be hybridized or annealed together.

A “marker” or “label” refers to any atom or molecule that may be used to provide a detectable (preferably quantifiable) effect and that can be attached to a nucleic acid. Markers may include colored dyes; radioactive labels; binding moieties such as biotin; haptens such as digoxigenin; luminogenic, phosphorescent or fluorogenic moieties; and fluorescent dyes alone or in combination with moieties that can suppress or shift emission spectra by the energy transfer of fluorescence. Markers may provide signals, which are detectable for example by fluorescence, radioactivity, colorimetry, gravimetry, X-ray diffraction or absorption, magnetism and enzymatic activity. A marker may be a charged moiety (positive or negative charge) or may also have a neutral charge. They may include or consist of nucleic acid or protein sequence. Preferred markers are fluorescent dyes.

A “target” or “probe molecule” refers to a nucleic acid molecule to be detected. Target nucleic acids may contain a sequence that has at least a partial complementarity with at least a probe oligonucleotide.

“Probes” or “probe molecules” refer to nucleic acids, which interact with/hybridize to a target nucleic acid to form a detection complex.

The term “signal probe” or “probe” relates to a probe molecule, which contains a detectable moiety, which are already outlined above.

The term “nucleic acid” is meant to comprise any sequence of deoxyribonucleotides, ribonucleotides, peptido-nucleotides, including natural and/or artificial nucleotides.

The expression “sample” is meant to include any specimen or culture of biological and environmental samples or nucleic acid isolated therefrom. Biological samples may be animal, including human, fluid, such as blood or urine, solid or tissue, alternatively food and feed products and ingredients such as dairy items, vegetables, meat and meat by-products. Environmental samples include environmental material such as surface matter, soil, water, industrial samples and waste, for example samples obtained from sewage plant, as well as samples obtained from food and dairy processing instruments, apparatus, equipment, utensils, disposable and non-disposable items. The sample may be used as such in the assay or may be subjected to a preliminary selection step, such as e.g. culturing the sample under conditions favoring or selecting for P. aeruginosa in said sample. Also, the nucleic acids contained in the sample may be isolated prior to performing the assay. In the presence of a multi-resistant P. aeruginosa in the sample the resulting nucleic acid sample will contain the target nucleic acid which may be isolated from the biological sample in any way known to the skilled person, including conventional isolation comprising lysis of the cellular material of the biological sample and isolation of DNA or RNA therefrom. In case the target nucleic acid is present in a low amount, the said nucleic acid may be subjected to PCR, preferably to a multiplex PCR, to specifically amplify the target nucleic acid prior to performing the assay.

A “nucleic acid sample” may be a polynucleotide or oligonucleotide of a variable length and is represented by a molecule comprising at least 5 or more deoxyribonucleotides, preferably about 10 to 1000 nucleotides, more preferably about 20 to 800 nucleotides and more preferably about 20 to 100 or even more preferred about 20 to 60. The exact size will depend on many factors, which in turn depend on the ultimate function or use of the oligonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another.

According to an embodiment, the present micro-array comprises a carrier or support on which in the form of a specific pattern nucleic acids are immobilized. Said nucleic acids comprise sequences specific for at least 8 determinants of P. aeruginosa. For a correct determination of the presence of multi-resistant P. aeruginosa in a sample a number of at least eight determinants have proven to yield a doubtless, non-ambiguous result. Since due to single nucleotide polymorphisms (SNPs) contained in a particular determinant, said determinant has to be characterized by more than one nucleic acid sequence, so that more than one capture probe is required for particular determinants to provide a detectable hybridisation event under stringent conditions. In consequence, also number of nucleic acid capture probes corresponding to known SNPs is attached to the surface of the carrier of the present micro-array to act as the capture molecule for the particular determinant, thereby allowing the individual and unambiguous detection of each SNP of said determinant. The different capture probes (for the different SNPs) for one particular determinant may be attached to the carrier (e.g. spotted) on one localized area or on different ones.

Said immobilized nucleic acids comprise sequences specific for at least 8 determinants of P. aeruginosa, which sequences are preferably randomly selected from the group consisting of mexR, mexT, nfxB, mucA, parC, gyrA, exoU, exoS, exoT, pse, oxa, imp, vim, aac, aph and aad. Each of these determinants is detected either by a single capture probe or a set of two or more capture probes, which number of capture probes depend from the number of SNPs said determinant embraces. For a correct and unambiguous identification of the strain and the detection of a multiresistant P. aeruginosa strain 8 determinants, which may include resistance genes and other genes conferring virulence to said strain, have proven to be sufficient without any requirements concerning the selection of the determinants. The detection of 9 or more determinants is preferred, since in this case more precise information about antibiotic determinants and other virulence factors are achieved. Thus, the present micro-array may also comprise nucleic acids probes specific for at least 9 to 11 determinants, more preferably at least 12 to 14 determinants, still more preferably at least 15 determinants and most preferably nucleic acids probes specific for 16 determinants.

The inclusion of P. aeruginosa specific control capture probes (oprI and gyrB) as well as capture probes for the detection of a broad range of gram-negative organisms (srv3) allows a more correct species identification.

The carrier or support of the present DNA micro-array may consist of different materials, preferably of glass, silicon, silica, metal, plastics or mixtures thereof prepared in format selected from the group of slides, discs, gel layers and/or beads. The carrier may also be a microplate or a slide and may consist of epoxy glass. A preferred support is for example an epoxy modified glass slide purchased by Elipsa AG, Berlin, Germany.

Preferably, the present micro-array has at least 100 molecules attached per square centimeter of the solid carrier. This density may be, however, higher and be adapted to the respective application of the micro-array, in that also other suitable applications may be performed, e.g. for the determination of resistances in other organisms different from P. aeruginosa and/or for the detection of resistance gene(s), which are unknown yet to play a role in P. aeruginosa. For example, the density of the nucleic acids probes attached per square centimeter of solid carrier amounts more preferably at least to 1.000, still more preferably at least to 5.000 and most preferably at least to 10.000 nucleotides per square centimeter.

Said specific pattern allows the mapping of each nucleic acid probe to a specific position on said carrier and a specific analysis, in that the analysis of the results of the present micro-array is facilitated and non-ambiguous concerning the attribution of a particular spot to a previous attached nucleic acid probe.

Spacer molecules of any length may be arranged between the carrier and the nucleic acids applied on the carrier. The spacer may be for example polymer-based spacers, but may also consist of an alkane chain, or any derivatives thereof, of a suitable length, which comprises at each end respective functional groups for attachment to the solid support and the nucleic acid probe. Preferably, 15-thymidine spacers have been attached with one end to the surface of the support and with the other end to the 3′-terminal end of the respective nucleic acid to be immobilized.

According to another preferred embodiment, the present invention provides a method for the detection of multi-resistant P. aeruginosa strains in a sample material, using a micro-array for the detection of determinants like resistance genes and other genes conferring virulence.

The method comprises the step to obtain a sample material of interest. Prior to performing the method of the present invention the sample may be pre-treated e.g. centrifuging or filtering to separate non-soluble matter or selecting for P. aeruginosa in the sample. This may be achieved by e.g. culturing the sample under conditions favouring the growth of P. aeruginosa. Also, to improve performance, nucleic acids contained in the sample material may be isolated and/or amplified. The sample and/or the isolated/purified nucleic acid material is applied to the surface of the present micro-array. Said sample is now allowed to hybridize to the immobilized nucleic acids, the capture probes, for targeting at least 8 determinants of P. aeruginosa. By choosing suitable hybridisation conditions known to the skilled person, such as e.g. applying a certain stringency during hybridization and washing (cf. Maniatis et al., Molecular Cloning—A Laboratory Manual, First Edition, Cold Spring Harbor, 1982), only those nucleic acids will hybridize to the immobilized nucleic acids and/or remain bound during washing steps, which exhibit a high homology to the immobilized nucleic acids. The method further comprises detecting any hybridisation event, which will be indicative of the presence of a multi-resistant P. aeruginosa.

Said nucleic acids probes specific for targeting at least 8 determinants of P. aeruginosa are preferably randomly selected from the group consisting of mexR, mexT, nfxB, mucA, parC, gyrA, exoU, exoS, exoT, pse, oxa, imp, vim, aac, aph and aad. Each of these determinants is detected by a specific set of capture probes, which may comprise more than one nucleic acid probe in accordance to the number of SNPs said determinant embraces. For a correct and non-ambiguous indentification of the strain and the determination of a multiresistant P. aeruginosa strain 8 determinants, which include resistance genes and other genes conferring virulence to said strain, have proven to be sufficient without any requirements concerning the selection of the determinants. Preferably, the micro-array may also comprise nucleic acids specific for at least 9 to 11 determinants, more preferably at least 12 to 14 determinants, still more preferably at least 15 determinants and most preferably 16 determinants.

P. aeruginosa specific control probes (oprI and gyrB) may be included. Other controls are probes, which are capable to detect a broad range of gram-negative organisms (srv3) for a correct species identification.

The nucleic acid sample to be used for hybridizing to the immobilized nucleic acids consists preferably of oligonucleotides and/or polynucleotides of a length between 10 and 1000 nucleotides each, preferably shorter oligonucleotides/polynucleotides exhibiting a length of about 10 to 100 or between 20 to 60. The length may be obtained for example by the digestion of plasmid or genomic DNA with DNAse or preferably restrictions enzymes and facilitates the hybridization.

The nucleic acid sample, which comprises oligonucleotides and/or polynucleotides, is preferably isolated from body tissues or fluids, particularly blood, suspected to contain P. aeruginosa, followed by the isolation and optional the amplification of the DNA and/or RNA contained therein by PCR techniques, such as a multiplex PCR, which allows the amplification of several DNA fragments in one PCR reaction. Such techniques are well known to the skilled person and may be also performed with commercial available kits.

The capture and the target nucleic acids may be present in a labeled form. The target nucleic acids may be labeled prior to performing the assay, by including a marker molecule into the molecule, e.g. during its amplification or isolation. Said marker molecule is preferably a fluorescent marker. Also the capture molecules may be labeled, in case of a fluorescent dye preferably with a dye exhibiting a different excitation and/or emittance wavelength, which allows a normalization of the experiment.

Methods for the detection of binding include e.g. surface plasmon resonance or detection of fluorescence at a localized area indicative of binding of a labelled molecule. Fluorescence may be detected e.g. via confocal laser induced fluorescence.

In another embodiment of the invention, a diagnostic kit is provided for the detection of P. aeruginosa infections.

Said kits either provides the nucleic acids specific for 16 determinants of P. aeruginosa, which determinants are selected from the group consisting of mexR, mexT, nfxB, mucA, parC, gyrA, exoU, exoS, exoT, pse, oxa, imp, vim, aac, aph and aad. Alternatively, the kit may also provide a micro-array as detailed above.

Additionally, the kit may also include the appropriate controls, in that probes are included specific for the gyrB, oprI and srv3 genes.

A typical automated processing of a micro-array according to a preferred embodiment of the present invention includes the use of three components. First, the micro-array or support respectively, second a reader unit and third means for the evaluation of the results, e.g. a suitable computer software. The reader unit comprises in general a movable tray, focusing lens(es), mirrors and a suitable detector, e.g. a CCD camera. The moveable tray carries the micro-array and may be moved to place the micro-array within the light path of one or more suitable light sources, e.g. a laser with an appropriate wavelength to excite a fluorescent compound. The evaluation program or software may serve for example to recognize specific patterns on the array or to analyze different expression profiles of genes. In this case, the software searches colored points on the array and compares the intensity of different color spectra of the same point. The result may be interpreted by an analyzing unit and afterwards stored in a suitable file format for further processing.

As detailed above, the probe- and/or target-nucleic acids may be labelled each with a fluorescent dye and the intensity of the fluorescence at different wavelengths of each point is compared to the background. The detector, e.g. a photomultiplier or CCD array, transforms low light intensities to an amplifiable electrical signal. Other methods use different enzymes, which are covalently bound to the nucleotide by means of a linker molecule. The enzymatic colorimetry uses for example alkaline phosphatase and horseradish peroxidase as marker. By contacting with a suitable molecule, a detectable dye may be achieved. Other chemoluminescent or fluorescent marker comprise proteins capable to emit a chemoluminescent or fluorescent signal, if irradiated with light of a discrete, specific wavelength, e.g. 488 nm for the green fluorescent protein. Radioactive markers are applied in case of low detection limits are required, but are due to their harmful properties not wide spread. Fluorescence marking is performed with nucleotides linked to a fluorescent chromophore. Combinations of nucleotides and fluorescent chromophore comprise in general Cy3 (cyanine 3)/Cy5 (cyanine 5) labelled dUTP as dye, since they may be easily incorporated, the electron migration for fluorescence may be exited by means of customary lasers and they also have distinct emission spectra.

The hybridisation of micro-arrays follows essentially the conventional conditions of southern or northern hybridisations, which are well known to the skilled person. The steps comprise a pre-hybridisation, the intrinsic hybridisation and a washing step after hybridisation occurred. The conditions have to be chosen in such a way that background signals are kept low, minimal cross-hybridisation (in general a reduced number of mismatches) occurs and with a sufficient signal strength, which has to be proportional for some applications to the concentration of the target molecule.

The hybridisation event may be detected generally by two different kinds of array-scanners. One method employs the principle of the confocal laser microscopy, which uses at least one laser to scan the array in point-to-point manner. Fluorescence is than detected by photomultipliers, which amplify the emitted light. The cheaper GGD basing readers use typically filtered white light for the excitation. The surface of the array is scanned with this method in sections, which allows the faster achievement of results of a lower significance.

Also the so-called gridding for the analysis of the results, in which an idealised model of the layout of the micro-array is compared with the scanned data to facilitate the spot definition. Pixels are classified (segmented) as spot (foreground) or background to produce the spotting mask. Segmentation techniques may be divided in fixed segmentation circle, adaptive circle segmentation, adaptive shape segmentation and histogram segmentation. The use of these techniques depends from the shape of the spots (regular, irregular) and the quality of the proximal arrangement of the spots.

Another issue is the intensity of the distinct spots, since the concentration of hybridised nucleotides in one spot is proportional to the total fluorescence of this spot. In particular, the overall pixel intensity and the ratio of the different fluorescent chromophores used (in case of Cy3 and Cy5, green and red) are important for the calculation of the spot intensity. Beneath the spot intensity, also the background intensity has to be taken into account, since various effects may disturb the fluorescence of the spots, for example the fluorescence of the support and of the chemicals used for the hybridisation. This may be performed by the so-called normalisation, which includes the above-mentioned effects and others like fluctuations of the light source, the lower availability/incorporation of the distinct marker molecules (Cy5 worse than Cy3) and their differences in emission intensities. Of importance for the normalisation is further the reference against which shall be normalized. In general, this may be a specific set of genes or a group of control molecules present on the micro-array.

The results may be further processed by means of the available software tools and according to the knowledge of bioinformatics.

The present invention provides a method, a micro-array and kit for the detection of P. aeruginosa infections, helpful in reducing the spread of multi-resistant isolates and improve the treatment options of severe and often life-threatening P. aeruginosa infections. The present inventors could verify surprisingly in the course of their studies also the presence of a vim gene in a clinical isolate, which represents the first alarming occurrence of said determinant in connection with a multi-resistant P. aeruginosa strain in Germany.

It is to be understood, that the above description is intended to be illustrative and not restrictive. Many embodiments will be apparent to those of skilled in the art upon reviewing the above description. By way of example, the invention has been described preliminary with reference to the use of nucleic acids comprising sequences specific for the resistance and virulence determinants of P. aeruginosa. It should be clear that also other resistance and virulence determinants may be selected in dependence from the genetic development of multiresistant P. aeruginosa strains. The scope of the invention should, therefore, be determined not with reference to the above description, but should instead be determined with reference to the appended claims, along with the full scope of equivalents to which such claims are entitled.

### EXAMPLES

### A. Bacterial Strains and Culture Conditions

The wild-type reference strain P. aeruginosa PAO1 was obtained from the ATCC (AT47085). The other P. aeruginosa strains were collected from patients at the Robert Bosch Hospital in Stuttgart, Germany. They were recovered from respiratory samples (n=51), swabs (n=5), urine (n=2) and faeces (n=2). All isolates were identified with the API 20NE system (bioMerieux, Marcy l'Etoile, France) and the NEG Breakpoint Combo Type 30 panel on the MicroScan WalkAway®-96 SI system (Dade Behring, Liederbach, Germany). All bacterial strains were either routinely cultured at 37° C. on Mueller-Hinton (MH) agar or grown in Luria Bertani broth (LB).

### B. Antibiotic Susceptibility Testing

The antibiotic susceptibility was determined with the NEG MIC Type 30 panel on the MicroScan WalkAway®-96 SI system. The MICs were interpreted according to the NCCLS guidelines. The strains were tested for aztreonam (AZT), ceftazidime (CAZ), cefepime (CPE), piperacillin (PI), piperacillin/tazobactam (P/T), imipenem (IMP), meropenem (MER), levofloxacin (LVX), ciprofloxacin (CP), colistin (COL), gentamicin (GM), tobramycin (TO) and amikacin (AK).

### C. DNA Methods, PCR, Labeling and Sequencing

Chromosomal DNA was extracted with the QIAmp DNA Mini Kit, plasmid DNA with the QIAprep Spin Miniprep Kit according to manufacturer's instructions (Qiagen, Hilden, Germany). A set of 4 multiplex PCRs (Tab. 1) was set up to amplify the sequences of interest from the chosen genes. The PCRs were carried out with the Advantage®-GC Genomic PCR Kit (BD Bioscience, San Jose, USA) according to manufacturer's instructions, for fluorescence labeling 5 μl of each 1 mM dNTP was used, for dCTP a 2:3 mixture of Cy3-dCTP and dCTP. The cycle reactions consisted of 30 cycles of 30 s at 95° C., 30 s at 55° C. and 1 min at 72° C. PCR products were purified with the QIAquick PCR Purification Kit (Qiagen, Hilden, Germany) according to the manufacturer's instructions. The quality and sizes of PCR products were checked on a lab-on-a-chip Bioanalyzer 2100 electrophoresis with the DNA 1500 LabChip kit (Agilent, Böblingen, Germany). Sequencing was performed on the ABI PRISM® 310 Genetic Analyzer using the BigDye® Terminator Cycle Sequencing Kit v 1.1 (Applied Biosystems, Foster City, USA). The manufacturer's protocol was followed using the same primers as for the multiplex PCRs. Sequences were assembled, aligned and analyzed with the Lasergene software, Version 5.08 (DNAStar, Madison, USA).

### D. Probe Design

Oligonucleotide probes specific for positions affected by SNPs were designed to have a nucleotide responsible for perfect match/mismatch at a central position. A set of 4 oligonucleotide probes was designed for each particular SNP having identical sequence except that for the central base, which was one of the 4 possible nucleotides A, T, G or C. A reliable detection of mutations due to insertion or deletion of particular bases was achieved with two probes optimized either for wild-type sequence or mutated sequence. The presence of relevant genes was confirmed with a set of two probes for each gene. Both probes were designed to represent the same sequence, one for sense direction and the other one for anti-sense direction. In order to keep all the probes within a certain thermal range for a simultaneously hybridization, the length of the capture oligonucleotides were varied between 17 and 24 bases.

### E. Array Fabrication

The oligonucleotide array consisted of 202 amino-modified capture probes containing a poly-(T) 15 spacer at the 5′-end. They were synthesized by MWG Biotech (Ebersberg, Germany) and resuspended in spotting buffer S1 (160 mM Na₂SO₄, 130 mM Na₂HPO₄) to a final concentration of 20 μM. The array layout is shown in FIG. 1. Each capture probe was spotted in triplicates on CreativeChip™ Oligonucleotide slides (Elipsa AG, Berlin, Germany) with the Microgrid II arraying system using MicroSpot 2500 pins (Biorobotics, Cambridge, UK). Spotted capture probes were covalently immobilized to the glass surface by incubation at 60° C. for 30 min in a drying compartment (Memmert, Schwabach, Germany). Blocking and cleaning of the fabricated slides until further use was performed according to the manufacturer's instructions.

### F. Controls.

Several controls were included on the array:

a spotting control (5′-cyanine 5 [Cy3]-TTTTTTTTTTTTTTCTAGACAGCCACTCATA-3′) (SEQ ID NO: 1);

a positive hybridization control (5′-TTTTTTTTTTTTTGATTGGACGAGTCAGGAGC-3′) (SEQ ID NO: 2) complementary to a labeled oligonucleotide target (5′-Cy3-GCTCCTGACTCGTCCAATC-3′) (SEQ ID NO: 3), which was spiked during hybridization; and

a negative hybridization control (5′-TTTTTTTTTTTTTTCTAGACAGCCACTCATA-3′) (SEQ ID NO: 4). All these control sequences are unrelated to sequences found in bacterial species.

### G. Fragmentation

In order to increase hybridization efficiency, the amplified and labelled target DNA was diluted to a concentration of 30 ng/μl in reaction buffer (40 mM Tris/HCl, pH 8.0, 10 mM MgSO₄, 1 mM CaCl₂) and fragmented with DNAseI (11.5 mU/μl) (Invitrogen, Karlsruhe, Germany) at room temperature for 5 min. The reaction was stopped by addition of 3 mM EGTA and incubation at 65° C. for 10 min.

### H. Hybridization

400 ng fragmented target DNA with addition of control DNA (0.05 pmol) were hybridized under a 18 mm×18 mm cover glass in 30 μl of 6×SSPE (1× is 0.18 M NaCl, 10 mM NaH₂PO₄, and 1 mM EDTA [ph 7.7]), incubated in an Eppendorf Thermomixer Comfort (Eppendorf AG, Hamburg, Germany) at 55° C. for 1 h. After hybridization the slides were washed with 2×SSC, 0.1% SDS then 2×SSC (1× is 0.15 M NaCl plus 0.015 M sodium citrate) and 0.2×SSC, each time for 10 min at room temperature with agitation in a glass container. Finally, the slides were dried with air.

### I. Data Acquisition and Processing

The oligonucleotide arrays were scanned with an arrayWoRx Biochip Reader (Applied Precision, Marlborough, UK). The scanner settings for fluorescence signal acquisition were set to “High Precision” and 0.2 s acquisition time. The image processing and calculation of signal intensity was performed with the ArrayPro software (MediaCybernetics, San Diego, USA). The net signals were obtained by subtraction of the local background from the absolute density signal. The global background area is defined as the area between the spots of the array. The software calculates a minimum, maximum and average background value from the global background data.

### J. Susceptibility Profile of the Isolates

The test collective of P. aeruginosa isolates was recovered from three intensive care units (ICU) or other hospital wards as far as an unusual multidrug resistance was observed. Most isolates were obtained from respiratory samples, followed by wound swabs and urine (Tab. 3). An overview of the antibiotic susceptibilities of the isolates is shown in Tab. 4. Susceptibility profiles of the tested strains showed a typical distribution for an ICU. Ten P. aeruginosa isolates showed a concomitant antibiotic resistance against one or more antibiotics from the group of aminoglycosides, fluoroquinolones, cephalosporines and carbapenemes. Among the tested substances colistin was most effective substance with a resistance rate of 0% and imipenem the endmost effective with a susceptibility rate less than 55%.

### K. Array Set-Up

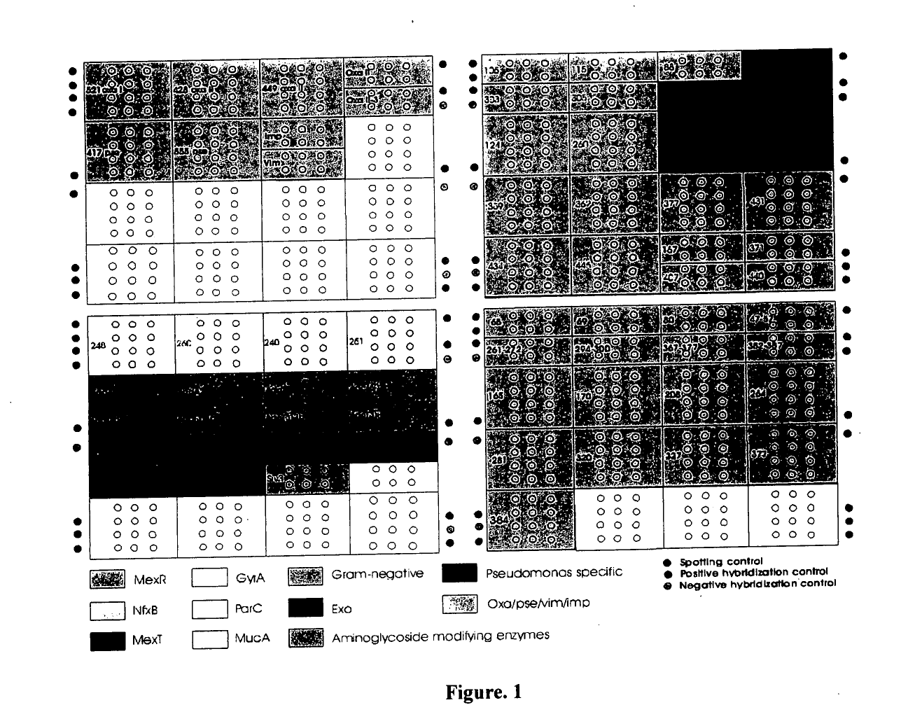

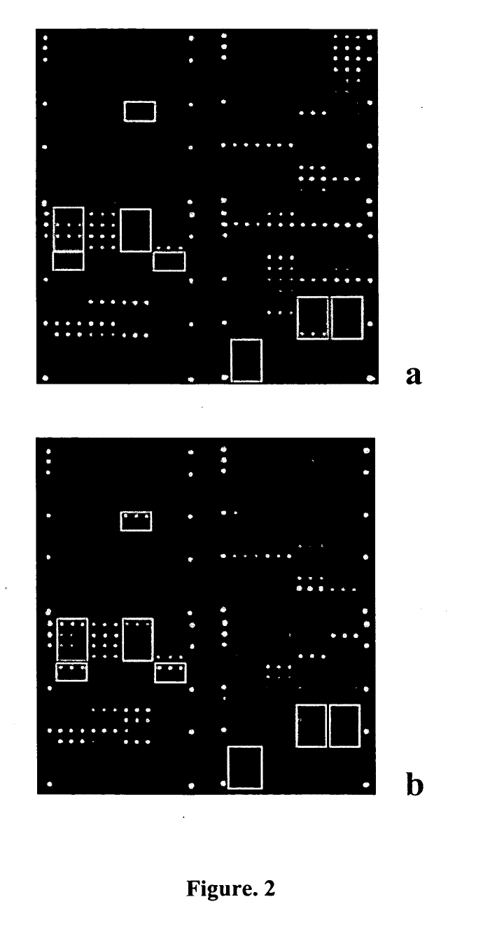

The two major features of array based test systems are sensitivity and specificity. In contrast to usually methods used to discriminate specific from unspecific signals based on an internal DNA standard we defined a cut-off value based on the background fluorescence intensity. The cut-off value for a specific, positive fluorescence signal was set to 1.5 times of the minimal background signal value. Each signal, regardless perfect match or mismatch was considered as positive, if the absolute fluorescence intensity exceeded this value. Everything below was considered as unspecific and was not considered in any subsequent analyzes. FIG. 2 shows a typical array experiment with hybridization patterns of the reference strain PAO1 (a) and P. aeruginosa isolate No. 23 (b). The hybridization of an array with target DNA obtained from PAO1 revealed a minimal background fluorescence intensity of 4.86×10⁹ RFU (relative fluorescence unit). The signal cut-off was set to 7.29×10⁹ RFUs. For P. aeruginosa isolate No. 23 the respective values were 5.06×10⁸ RFU and 7.59×10⁸ RFU.

The second major feature of an array is specificity defined by the ability to discriminate between mismatch and perfect match signal. The highest fluorescence signal of each SNP or insertion/deletion position was considered as potential perfect match position (PM). The other signals of probes specific for particular mutation position were considered as potential mismatch positions (MM) and normalized to the PM value. The potential perfect match signal was set to the value of 1.0 and the mismatch signals were adjusted accordingly. The ratio of MM/PM ranged from 0.0 to 1.0. The relative intensity value of particular probe was considered as specific, if MM/PM ratio for that probe did not exceed the value of 0.7. The MM/PM ratio for all of tested capture probes remained under 0.7.

Every gene which is subject to mutations was sequenced for both strands in order to determine the correlation between array deduced genotype and sequencing based genotype. In all cases, the highest array signals for the different SNP, insertion or deletion positions corresponded to the perfect match position according to the underlying genotype determined by sequence analysis. Also the presence of plasmid or integron encoded resistance genes not subject to mutations could be verified via PCR and sequencing analysis.

### L. Genotype Analysis By Array

The differential comparison of the two hybridization patterns showed a discrepant perfect match signal for positions 327, 377 and 384 in the mexR gene, position 248 in the gyrA and 240 in the parC gene (FIG. 3). All differences in perfect match signal were a matter of single nucleotide polymorphism. The mutations in mexR at position G327A and G384A were silent and did not cause an amino acid exchange, but mutation T377A causing a Val126Glu exchange was responsible for respective antibiotic resistance. The mutation C248T (Ser83Leu) in the gyrase gene gyrA as well as mutation C240T (Ser80Leu) found in the topoisomerase IV gene parC leads to respective amino acid substitution that is responsible for fluoroquinolone resistance. In addition, specific fluorescence signals from isolate No. 23 indicated the presence of a vim, aac(6′)-Ib and aadA1 gene. There were no additional differences as analyzed by the presented array in genotypical characteristics of clinical isolate No. 23 and reference P. aeruginosa strain PAO1. For oprI, srv3, exoS, exoT, aadA1 and aac(6′)-Ib the signals for sense and anti-sense probes are displayed, showing that usually one get signals above the cut-off for both probes.

The 60 P. aeruginosa isolates were tested with the array applying the same hybridization conditions. Tab. 4 and Tab. 5 show a summary of the array analysis, covering the genes which are affected by mutations or are acquired by plasmid acquisition. The presence of such genes may contribute to antibiotic resistance or virulence.

The distribution of the mutations indicate the existence of hot spots at sequence positions 327, 377 and 384 for the mexR gene, 305 for the nfxB gene, 197 and 212 for mexT and 248 for the gyrA gene, respectively. 22 isolates harbored 3 mutations in mexR at position 327, 377, 384. Furthermore, the array analysis of 60 P. aeruginosa clinical isolates revealed as well the presence of plasmids or integrons encoding for antibiotic resistance genes (imp, vim, oxa, aad, aac). Concerning the group of aminoglycoside modifying enzymes, the following genes were detected: aac(6′)-Ib, aac(3)-Ia, -Ib and -II, aadA1, -2, aadB and aph(3′). In the group of β-lactamases, 8 imp and one vim-1 gene could be determined. However, the most frequent β-lactamase genes found in 17 isolates of the collective belong to the oxa-family, 13 of them the ESBL oxa-14 gene. The array data revealed the presence of virulence factors, exoS and exoT genes were found in 47 isolates, 13 isolates harbored an exoU gene. This is consistent with the usual frequencies reported for these genes. ExoS and exoT were reported to occur with a frequency of about 68%, exoU with 28%, respectively (78% and 22% respectively in our collective).

### M. Interarray Variability

In order to determine if the ratio of perfect match to mismatch signal intensity after hybridization with target DNA from a set of different amplification/labeling reactions is reproducible, the array was hybridized four times with DNA obtained from PAO1. However, due to different dye incorporation and fragmentation efficiency the net intensities varied for some positions for a factor higher than three. Therefore, the relative intensity was calculated. After normalization, the overall interarray variability was 8.7%. In any case, the relation between the 4 probes specific for a single SNP position was not changed from an array to another.

### O. Array Signal—Genotype Correlation

Every gene, which is subjected to mutations was sequenced for both strands in order to determine the correlation between array deduced genotype and sequencing based genotype. In all cases, the highest array signals for the different SNP, insertion or deletion positions corresponded to the perfect match position according to the underlying genotype determined by sequence analysis. Also the presence of plasmid or integron encoded resistance genes not subject to mutations could be verified via PCR and sequencing analysis.

### P. Correlation of Array Based Genotype and Phenotype

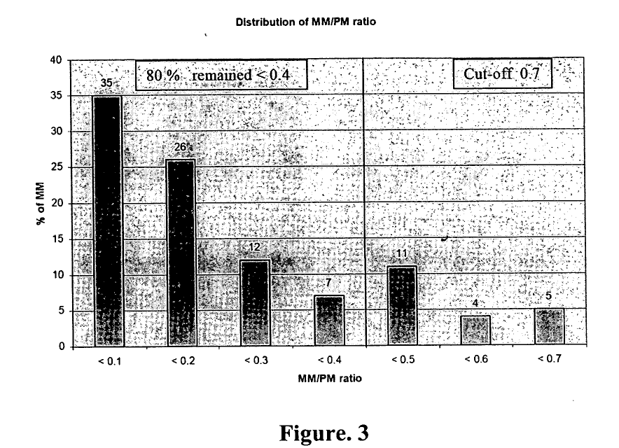

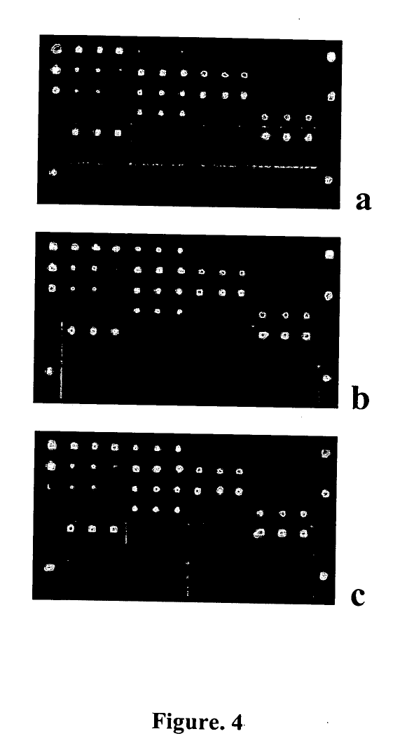

The FIG. 4 shows a detail of the array analyses of three P. aeruginosa isolates that were collected from the same patient within a time period of three weeks. The first isolate was phenotypically susceptible to ciprofloxacin and tobramycin. The subsequent isolate, collected one week after the first isolate, was phenotypically already resistant against ciprofloxacin but still susceptible for tobramycin. Finally, the last of the three isolates then showed phenotypical resistance for ciprofloxacin and tobramycin. A retrospective array analysis of these three isolates correlated well with the resistance phenotype. However, due to higher sensitivity it was possible to detect resistance relevant genes and mutations even in the first isolate (FIG. 4). All isolates carried a mutation at position 248 in gyrA (levofloxacin and ciprofloxacin resistance) and harbored an aac(6′)-Ib (tobramycin and amikacin resistance) and aadA1 gene (gentamicin and tobramycin resistance).

In order to compare genotype and phenotype based resistance profiles, we calculated a cumulative resistance rate against different groups of antibiotics based on the array data (Tab. 6). Since one isolate may harbor one or more mutations or resistance genes which may confer resistance to a certain antibiotic at the same time, the cumulative resistance rates predicted from our array data were 5 to 10% higher as compared with the phenotypic determined susceptibility rates.

TABLE 1
Primers used for amplification

| | | | | | GenBank | Multi- |
|---|---|---|---|---|---|---|
| | Covered | | | Product | accession | plex |
| Name | genes | Primer | Sequence (5′-3′) | Size | number | PCR |
| MexR | mexR | MexR for | GATGCCCGCGCTGATGG (SEQ ID NO: 5) | 390 | AE004479 | I |
| | | MexR rev | AGGCACTGGTCGAGGAGATG (SEQ ID NO: 6) | | | |
| MexT | mexT | MexT for | ATGCCTGTCAGTGATCCTATGC (SEQ ID NO: 7) | 935 | AE004676 | |
| | | MexT rev | CGGGTCTCGAACGGTGGGTCCTC (SEQ ID NO: 8) | | | |
| NfxB | nfxB | NfXB for | GCGACGCTGAAGGAACTGG (SEQ ID NO: 9) | 240 | AE004874 | |
| | | NfXB rev | CCGGGCGGTACTGGAATA (SEQ ID NO: 10) | | | |
| MucA | mucA | MucA for | CAGCTTGCGGCGAGGATGC (SEQ ID NO: 11) | 454 | AE004511 | |
| | | MucA rev | GTACCACTGACGGCGGATTGTTGC (SEQ ID NO: 12) | | | |
| parC | parC | parC for | CTGGATGCCGATTCCAAGCAC (SEQ ID NO: 13) | 186 | AB003428 | |
| | | parC rev | GAAGGACTTGGGATCGTCCGG (SEQ ID NO: 14) | | | |
| gyrA | gyrA | gyrA for | GACGGCCTGAAGCCGGTGCAC (SEQ ID NO: 15) | 417 | AE004741 | |
| | | gyrA rev | GCCCACGGCGATACCGCTGGA (SEQ ID NO: 16) | | | |
| ExoU | exoU | ExoU for | CCGTTGTGGTGCCGTTGAAG (SEQ ID NO: 17) | 135 | U97065 | II |
| | | ExoU rev | CCAGATGTTCACCGACTCGC (SEQ ID NO: 18) | | | |
| ExoU | exoU | ExoU for | CCGTTGTGGTGCCGTTGAAG (SEQ ID NO: 19) | 135 | U97065 | |
| | | ExoU rev | CCAGATGTTCACCGACTCGC (SEQ ID NO: 20) | | | |
| ExoS | exoS | ExoS for | GCGAGGTCAGCAGAGTATCG (SEQ ID NO: 21) | 119 | AE004801 | |
| | | ExoS rev | TTCGGCGTCACTGTGGATGC (SEQ ID NO: 22) | | | |
| ExoT | exoT | ExoT for | AATCGCCGTCCAACTGCATGCG (SEQ ID NO: 23) | 154 | AE004444 | |
| | | ExoT rev | TGTTCGCCGAGGTACTGCTC (SEQ ID NO: 24) | | | |
| GyrB | gyrB | GyrB for | CCTGACCATCCGTCGCCACAAC (SEQ ID NO: 25) | 329 | AB005881 | |
| | | GyrB rev | CGCAGCAGGATGAAGACGCC (SEQ ID NO: 26) | | | |
| OprI | oprI | OprI for | GCTCTGGCTCTGGCTGCT (SEQ ID NO: 27) | 197 | AE004712 | |
| | | OprI rev | AGGGCACGCTCGTTAGCC (SEQ ID NO: 28) | | | |
| Srv3 | 16S | Srv3 for | CGGNCCAGACTCCTACGGG (SEQ ID NO: 29) | 204 | AE004949 II | |
| | rRNA | Srv3 rev | TTACCGCGGCTGCTGGCA (SEQ ID NO: 30) | | | |
| Pse | carb- | Pse for | GCTAAATTACTATGATGCTGAG (SEQ ID NO: 31) | 327 | S46063, | |
| | 1,2,3,4 | Pse rev | TATTGCCTTAGGAGTTGTCG (SEQ ID NO: 32) | | U14749 | |
| Oxa | Oxa- | OxaI for | CAGAGAAGTTGGCGAAGTAAGAAT (SEQ ID NO: 33) | 307 | AF347074, | III |
| | 5,7,10,11, | OxaI rev | AACCCACCCAACCCACCAT (SEQ ID NO: 34) | | U37105, | |
| | 13,14,16, | | | | Z22590, | |
| | 17,19,28 | | | | U59183 | |
| | | | | | L38523, | |
| | | | | | AF043100, | |
| | | | | | AF060206, | |
| | | | | | AF043381, | |
| | | | | | AF231133 | |
| | oxa- | OxaII for | GCTCGGCGCTATTTGAAGAA (SEQ ID NO: 35) | 415 | AJ295229, | |
| | 2,3,15,20 | OxaII rev | GCGCAGCGTCCGAGTTGA (SEQ ID NO: 36) | | L07945, | |
| | | | | | U63835, | |
| | | | | | AF024602 | |
| Imp | Imp- | Imp for | GACACTCCATTTACTGCTA (SEQ ID NO: 37) | 160 | X98393, | |
| | 1,7,9,10, | Imp rev | ATTCAGATGCATACGTGGGGATAG (SEQ ID NO: 38) | | AY625689, | |
| | 11 | | | | AF318077, | |
| | | | | | AY033653, | |
| | | | | | AB074434, | |
| | | | | | AB074437,A | |
| | | | | | B074433 | |
| Vim | Vim-1,2 | Vim for | TGATACAGCGTGGGGTGCGAAAAA (SEQ ID NO: 39) | 472 | AJ291609, | |
| | | Vim rev | GTGCCCCGGAATGACGAACTGTG (SEQ ID NO: 40) | | AF263519, | |
| | | | | | AJ295229 | |
| Aac | Aac(6′)-Ib | Aac(6′)-Ib for | CTCGAATGCCTGGCGTGTTTGA (SEQ ID NO: 41) | 439 | X60321, | IV |
| | | Aac(6′)-Ib rev | GTGGTGGGGCGGAGAAGAAGC (SEQ ID NO: 42) | | AF043381, | |
| | | | | | U59183, | |
| | | | | | AF231133, | |
| | | | | | AF315351, | |
| | | | | | AF315786 | |
| | Aac(6′)-II | Aac(6′)-II for | ACTGGTCTATTCCTCGCACTCCTG (SEQ ID NO: 43) | 288 | AF162771, | |
| | | Aac(6′)-II rev | CCCCCATAACTCTTCGCCTCAT (SEQ ID NO: 44) | | M29695, | |
| | | | | | AF318077 | |
| | Aac(3)-Ia | Aac(3)-Ia for | GCCGGAGACTGCGAGAT (SEQ ID NO: 45) | 241 | U12338 | |
| | | Aac(3)-Ia rev | GCAGTCGCCCTAAAACAAA (SEQ ID NO: 46) | | | |
| | Aac(3)-Ib | aac(3)-Ib for | ACGCTTCAGGTGGCTAATC (SEQ ID NO: 47) | 345 | L06157 | |
| | | aac(3)-Ib rev | ACAAAGTTAGGTGGCTCAATG (SEQ ID NO: 48) | | | |
| | Aac(3)-II | aac(3)-II for | TTCCCCCAAGGCGTGACC (SEQ ID NO: 49) | 424 | AF466526 | |
| | | aac(3)-II rev | GCATACGCGGAAGGCAATAAC (SEQ ID NO: 50) | | | |
| Aph | Aph(3′)- | Aph(3′)-IIb for | GAAGAACTCGTCCAATAGCCTGAA (SEQ ID NO: 51) | 224 | X90856 | |
| | IIb | Aph(3′)-llb rev | GCGACGCCTGCCTGCCAAATC (SEQ ID NO: 52) | | | |
| Aad | aadA1 | AadA1 for | TATCAGAGGTAGTTGGCGTCAT (SEQ ID NO: 53) | 440 | AJ291609, | |
| | | AadA1 rev | TTCAGGAACCGGATCAAAGAGT (SEQ ID NO: 54) | | AJ295229 | |
| | aadA2 | AadA2 for | TCAGGAACCGGGTCAAAGAAT (SEQ ID NO: 55) | 416 | U12338 | |
| | | Aada2 rev | GAGCGCCATCTGGAATCAAC (SEQ ID NO: 56) | | | |
| | aadB | AadB for | CGGCACGCAAGACCTCAA (SEQ ID NO: 57) | 241 | AF078527, | |
| | | AadB rev | GCTTGGTGGGCAGACGAA (SEQ ID NO: 58) | | AF133699 | |
| | | | | | IV | |

TABLE 2
Origin and source of the test collective
of 60 clinical P. aeruginosa isolates

| | ICU- | ICU- | ICU- | | |
|---|---|---|---|---|---|
| | Internal | General | Cardiac | Other | |
| | Medicine | Surgery | Surgery | wards | All |
| Bronchoalveolar | | | | 1 | 1 |
| lavage | | | | | |
| Tracheal | 5 | 9 | 18 | 1 | 33 |
| sectretion | | | | | |
| Swab | 2 | 2 | 4 | 2 | 10 |
| Pharynx/Nose | | | | | |
| Sputum | 3 | | | 4 | 7 |
| Swab Wound | 1 | 3 | | 1 | 5 |
| Faeces | 1 | | | 1 | 2 |
| Urine | | | | 2 | 2 |
| Total | 12 | 14 | 22 | 12 | 60 |

TABLE 3
Antimicrobial susceptibilities of P. aeruginosa test collective

| | Number of | % of resistant |
|---|---|---|
| | resistant isolates | isolates out |
| Antibiotic | out of 60 | of 60 |
| Levofloxacin | 26 | 43 |
| Ciprofloxacin | 24 | 40 |
| Gentamicin | 22 | 37 |
| Tobramycin | 6 | 10 |
| Amikacin | 10 | 17 |
| Piperacillin | 21 | 35 |
| Tazobac | 20 | 33 |
| Ceftazidim | 20 | 33 |
| Cefepim | 19 | 32 |
| Aztreonam | 22 | 36 |
| Imipenem | 27 | 45 |
| Colistin | 0 | 0 |

TABLE 4
Mutations found by array analysis in 60 P. aeruginosa isolates

| | Gene | | | | | | | | | | | | | |
|---|---|---|---|---|---|---|---|---|---|---|---|---|---|---|
| | mexR | | | | | nfxB | | | mexT | | gyrA | parC | mucA | |
| Mutation locus | 80 | 208 | 327 | 377 | 384 | 115 | 303 | 305 | 197 | 212 | 248 | 240 | 362 | 440 |
| Number of | 2 | 6 | 24 | 24 | 22 | 1 | 3 | 9 | 13 | 10 | 19 | 3 | 2 | 4 |
| isolates out of 60 | | | | | | | | | | | | | | |

TABLE 5
Antibiotic resistance mediating genes found by
array analysis in 60 P. aeruginosa isolates

| | Gene | | | | | | | | | | | | | |
|---|---|---|---|---|---|---|---|---|---|---|---|---|---|---|
| | | | | aadA | | | Aac(3) | | | Aac(6′) | | | | |
| Subtype | Imp | Vim | Oxa | 1 | 2 | aadB | Ia | Ib | II | Ib | II | exoS | exoT | exoU |
| Number of | 8 | 1 | 17 | 5 | 1 | 4 | 4 | 2 | 7 | 6 | 0 | 47 | 47 | 13 |
| isolates | | | | | | | | | | | | | | |

TABLE 6
Comparison of phenotypic resistance rate and cumulative resistance
prediction based on genotype determined by array analysis

| | Number of | | | Cumulative |
|---|---|---|---|---|
| | resistant | % of | | resistance |
| | isolates | resistant | | predicttion |
| | out | isolates out | 60 MIC | deduced |
| Antibiotic | of 60 | of 60 | [μg/ml] | from array |
| Levofloxacin | 26 | 43 | >4 | 52% |
| Ciprofloxacin | 24 | 40 | >2 | |
| Gentamicin | 22 | 37 | >8 | 45% |
| Tobramycin | 6 | 10 | >8 | 17% |
| Amikacin | 10 | 17 | >32 | 20% |
| Piperacillin | 21 | 35 | >64 | 38% |
| Tazobac | 20 | 33 | >64 | |
| Ceftazidim | 20 | 33 | >16 | 40% |
| Cefepim | 19 | 32 | >16 | 40% |
| Aztreonam | 22 | 36 | >16 | |
| Imipenem | 27 | 45 | >8 | 50% |
| Colistin | 0 | 0 | | 0% |

TABLE 7
Susceptibility profile for ciprofloxacin and tobramycin from 3 consecutive
Pseudomonas aeruginosa isolates from the same patient

| Isolate 1 | Isolate 2 | Isolate 3 | Antibiotic |
|---|---|---|---|
| S | R | R | Ciprofloxacin |
| S | S | R | Tobramycin |

TABLE 8
sequences of the capture probes and the positions of the respective SNPs

| | Sequence | | Wild | | | |
|---|---|---|---|---|---|---|
| | position | | type | Mutation | Description | Length |
| Gene | (Name)* | Probe sequence** (5′-3′) | N*** | N**** | ***** | [bases] |
| mexR | 165 mexR se | tcccaggtcccNcaggttcag (SEQ ID NO: 59) | C | G | Gln55His | 21 |
| | 170 mexR se | tggcgtcccNggtcctgcag (SEQ ID NO: 60) | A | C | Leu57Arg | 20 |
| | 208 mexR se | ggatcttccNggtgatcagt (SEQ ID NO: 61) | G | A | Arg70Gln | 20 |
| | 264 mexR se | cgctggtccNtggggttg (SEQ ID NO: 62) | C | G | Ser88Arg | 18 |
| | 281 mexR se | aggaagagcNggaagctgcg (SEQ ID NO: 63) | T | G | Gln94Pro | 20 |
| | 320 mexR se | gcctccgcaNgctggtggat (SEQ ID NO: 64) | T | G | His107Pro | 20 |
| | 327 mexR se | atgatggcNtccgcatgctg (SEQ ID NO: 65) | C | A | Glu109Asp | 20 |
| | 377 mexR se | cttgttccNccggggtgag (SEQ ID NO: 66) | T | A | Val126Glu | 19 |
| | 384 mexR se | cagggtggcNtgttcctcc (SEQ ID NO: 67) | C | A | Gln128His | 19 |
| | 65 mexR se | ctggatgcgccggtccgcac (SEQ ID NO: 68) | | CG ins | frameshift/ | 20 |
| | | | | | stop | |
| | 65 mexR WT se | ctggatgcgcgtccgcac (SEQ ID NO: 69) | | | wild-type | 18 |
| | 69 mexR se | cgctctggatggcgcgtccg (SEQ ID NO: 70) | | C ins | frameshift/ | 20 |
| | | | | | stop | |
| | 69 mexR WT se | cgctctggatgcgcgtccg (SEQ ID NO: 71) | | | wild-type | 19 |
| | 80 mexR se | caatcgagcatcgctctgga (SEQ ID NO: 72) | | T ins | frameshift/ | 20 |
| | | | | | stop | |
| | 80 mexR WT se | caatcgagctcgctctgga (SEQ ID NO: 73) | | | wild-type | 19 |
| | 47-57 mexR se | gcgtccgcacccgccatcag (SEQ ID NO: 74) | | Δ10N | frameshift/ | 20 |
| | | | | | stop | |
| | 47-57 mexR WT se | tgctggaagaccgccatcag (SEQ ID NO: 75) | | | wild-type | 20 |
| | 261-272 mexR se | ctggaagctggggttgcgct (SEQ ID NO: 76) | | Δ10N | frameshift | 20 |
| | 261-272 mexR WT se | gtcgctggggttgcgctcg (SEQ ID NO: 77) | | | wild-type | 19 |
| | 294-300 mexR se | ggatggccagcccgtgagga (SEQ ID NO: 78) | | Δ10N | frameshift | 20 |
| | 294-3 00 mexR WT se | cagcccctcgtcggtgag (SEQ ID NO: 79) | | | wild-type | 18 |
| | 367-377 mexR se | ggcctgttcccggggcaaac (SEQ ID NO: 80) | | Δ10N | frameshift/ | 20 |
| | | | | | stop | |
| | 367-377 mexR WT se | ggggtgagcgggcaaaca (SEQ ID NO: 81) | | | wild-type | 18 |
| | 382-387 mexR se | gcaccagggtttccaccggg (SEQ ID NO: 82) | | Δ10N | frameshift | 20 |
| | 382-387 mexR WT se | accagggtggcctgttccac (SEQ ID NO: 83) | | | wild-type | 20 |
| mexT | 197 mexT se | cgcgggtcaNgctgcgttcg (SEQ ID NO: 84) | G | C | Leu65Val | 20 |
| | 212 mexT se | acagtttctNtgcggcgcgg (SEQ ID NO: 85) | C | T | Glu71Lys | 20 |
| | 733 mexT se | aggtcgccggNgaaggacacca (SEQ ID NO: 86) | C | A | Ala245Ser | 22 |
| | 413 mexT se | atcaatagaagttggcgcg (SEQ ID NO: 87) | | ΔT | frameshift/ | 19 |
| | | | | | stop | |
| | 413 mexT WT se | atcaatagatagttggcgcg (SEQ ID NO: 88) | | | wild-type | 20 |
| nfxB | 124 nfxB se | ccgcagaagcNgtgcagggt (SEQ ID NO: 89) | G | T | Arg41Ser | 20 |
| | 260 nfxB se | tgggtgaggNgttccttgat (SEQ ID NO: 90) | T | G | His87Pro | 20 |
| | 105 nfxB se | ggccttgctctacgccggccg (SEQ ID NO: 91) | | C ins | frameshift | 21 |
| | 105 nfxB WT se | ggccttgcttacgccggccg (SEQ ID NO: 92) | | | wild-type | 20 |
| | 115 nfxB se | ggtgcagggggccttgct (SEQ ID NO: 93) | | ΔT | frameshift/ | 18 |
| | | | | | stop | |
| | 115 nfxB WT se | ggtgcagggtggccttgct (SEQ ID NO: 94) | | | wild-type | 19 |
| | 188 nfxB se | tgatctggttccagtacgg (SEQ ID NO: 95) | | C ins | frameshift | 19 |
| | 188 nfxB WT se | tgatctggttcagtacgg (SEQ ID NO: 96) | | | wild-type | 18 |
| | 303 nfxB se | tccgggcggactggaatacc (SEQ ID NO: 97) | | ΔT | frameshift | 20 |
| | 303 nfxB WT se | tccgggcggtactggaatacc (SEQ ID NO: 98) | | | wild-type | 21 |
| | 305 nfxB se | tccgggcgggtactggaata (SEQ ID NO: 99) | | G ins | frameshift | 20 |
| | 305 nfxB WT se | tccgggcggtactggaata (SEQ ID NO: 100) | | | wild-type | 19 |
| gyrA | 248 gyrA A | tagaccgcgNtgtcgccgtg (SEQ ID NO: 101) | G | A | Ser83Ile | 20 |
| | 260 gyrA A | cacgatggtgNcgtagaccgc (SEQ ID NO: 102) | T | G | Asp87Tyr | 21 |
| parC | 240 parC A | tagcaggccNagtcgccgtg (SEQ ID NO: 103) | G | A | Ser80Leu | 20 |
| | 251 parC A | ccatggcctNgtagcaggcc (SEQ ID NO: 104) | C | T | Glu84Lys | 20 |
| exoS | exoS se | cttcaccaggccatccgc (SEQ ID NO: 105) | | | | 18 |
| | exoS as | gcggatggcctggtgaag (SEQ ID NO: 106) | | | | 18 |
| exoU | exoU se | gaaatcaccgcgctcgc (SEQ ID NO: 107) | | | | 17 |
| | exoU as | gcgagcgcggtgatttc (SEQ ID NO: 108) | | | | 17 |
| exoT | exoT se | aagtgctccaccaggccatc (SEQ ID NO: 109) | | | | 20 |
| | exoT as | gatggcctggtggagcactt (SEQ ID NO: 110) | | | | 20 |
| oprI | oprI se | cgtcttcggtagcggtcag (SEQ ID NO: 111) | | | | 19 |
| | oprI as | ctgaccgctaccgaagacg (SEQ ID NO: 112) | | | | 19 |
| gyrB | gyrB se | ctgaagtggatgttgctgaaggtc (SEQ ID NO: 113) | | | | 24 |
| | gyrB as | gaccttcagcaacatccacttcag (SEQ ID NO: 114) | | | | 24 |
| 16S rDNA | srv3 se | ttactgcccttcctcccaactta (SEQ ID NO: 115) | | | | 19 |
| | srv3 as | taagttgggaggaagggcagtaa (SEQ ID NO: 116) | | | | 19 |
| mucA | 359 mucA se | gtcccctgttNcgccatttgc (SEQ ID NO: 117) | C | T | Thr120Asn | 21 |
| | 362 mucA se | gtggtcccctNttgcgccatt (SEQ ID NO: 118) | C | T | Thr121Asn | 21 |
| | 377 mucA se | agggcgatcNgcggggtggtc (SEQ ID NO: 119) | C | T | Leu126Gln | 21 |
| | 431 mucA se | gccccctgctNttcgctgtag (SEQ ID NO: 120) | C | T | Ala144Glu | 21 |
| | 434 mucA se | ggcgccccctNctcttcgctg (SEQ ID NO: 121) | C | T | Pro145Gln | 21 |
| | 446 mucA se | gtgatcacctNcggcgccccc (SEQ ID NO: 122) | C | T | Thr149Asn | 21 |
| | 167 mucA se | agggtaggctccgcggtgcat (SEQ IS NO: 123) | | C ins | frameshift/ | 21 |
| | | | | | stop | |
| | 167 mucA WT se | agggtaggctcgcggtgcat (SEQ ID NO: 124) | | | wild-type | 20 |
| | 371 mucA se | atctgcggggggtcccctgt (SEQ ID NO: 125) | | ΔT | frameshift/ | 20 |
| | | | | | stop | |
| | 371 mucA WT se | atctgcggggtggtcccctgt (SEQ ID NO: 126) | | | wild-type 21 | |
| | 407 mucA se | ccagcacggcacgggcctttc (SEQ ID NO: 127) | | A ins | frameshift/ | 21 |
| | | | | | stop | |
| | 407 mucA WT se | ccagcacggccgggcctttc (SEQ ID NO: 128) | | | wild-type | 20 |
| | 440 mucA se | acctgcggcgcccctgctct (SEQ ID NO: 129) | | ΔC | frameshift/ | 20 |
| | | | | | stop | |
| | 440 mucA WT se | acctgcggcgccccctgctct (SEQ ID NO: 130) | | | wild-type 21 | |
| | 471 mucA se | cgggtatcgcatggacgagga (SEQ ID NO: 131) | | A ins | frameshift/ | 21 |
| | | | | | stop | |
| | 471 mucA WT se | cgggtatcgctggacgagga (SEQ ID NO: 132) | | | wild-type | 20 |
| aph(3′) | aph(3′)-IIb se | gaagaactcgtccaatagcctgaa (SEQ ID NO: 133) | | | | 24 |
| | aph(3′)-IIb as | ttcaggctattggacgagttcttc (SEQ ID NO: 134) | | | | 24 |
| aadB | aadB se | atgtgctttgtaggccagtcca (SEQ ID NO: 135) | | | | 22 |
| | aadB as | tggactggcctacaaagcacat (SEQ ID NO: 136) | | | | 22 |
| aadA | aadA1 se | tttcatcaagccttacggtcacc (SEQ ID NO: 137) | | | | 23 |
| | aadA1 as | ggtgaccgtaaggcttgatgaaa (SEQ ID NO: 138) | | | | 23 |
| | aadA2 se | ggtgacttctatagcgcggagc (SEQ ID NO: 139) | | | | 22 |
| | aadA2 as | gctccgcgctatagaagtcacc (SEQ ID NO: 140) | | | | 22 |
| aac(6′) | aac(6′)-Ib se | catacccaatcggctctccat (SEQ ID NO: 141) | | | | 21 |
| | aac(6′)-Ib as | atggagagccgattgggtatg (SEQ ID NO: 142) | | | | 21 |
| | aac(6′)-II se | aacgatgtgcggccggt (SEQ ID NO: 143) | | | | 17 |
| | aac(6′)-II as | accggccgcacatcgtt (SEQ ID NO: 144) | | | | 17 |
| aac(3) | aac(3)-II se | gttattgccttccgcgtatgc (SEQ ID NO: 145) | | | | 21 |
| | aac(3)-II as | gcatacgcggaaggcaataac (SEQ ID NO: 146) | | | | 21 |
| | aac(3)-Ib se | ctattgctgttccgcggtca (SEQ ID NO: 147) | | | | 20 |
| | aac(3)-Ib as | tgaccgcggaacagcaatag (SEQ ID NO: 148) | | | | 20 |
| | aac(3)-Ia se | aactcacgaccgaaaagatcaaga (SEQ ID NO: 149) | | | | 24 |
| | aac(3)-Ia as | tcttgatcttttcggtcgtgagtt (SEQ ID NO: 150) | | | | 24 |
| pse | 417 pse se | atcacttgtNgtcatagttg (SEQ ID NO: 151) T | A | pse-1,2,3 | 20 | |
| | | | | C | pse-4 | |
| | 555 pse se | caaatcaccNagcttaccttca (SEQ ID NO: 152) | T | A | pse-4 | 22 |
| | | | | C | pse-1,2,3 | |
| bla-imp | imp se | gagaattaagccactctattcc (SEQ ID NO: 153) | | | imp-1,7,9, | 22 |
| | | | | | 10,11 | |
| | imp as | ggaatagagtggcttaattctc (SEQ ID NO: 154) | | | imp-1,7,9, | 22 |
| | | | | | 10,11 | |
| bla-vim | vim se | atcaacgccgccgacgc (SEQ ID NO: 155) | | | vim-1,2,4 | 17 |
| | vim as | gcgtcggcggcgttgat (SEQ ID NO: 156) | | | vim-1,2,4 | 17 |
| oxa | 428 oxa1 se | gatattctggNtgccatagga (SEQ ID NO: 157) | A | T | oxa-10,13,14, | 21 |
| (group | | | | C | 16,17,19,28 | |
| 1) | | | | | oxa-11 | |
| | 521 oxal se | atttatttaaaNatagagactc (SEQ ID NO: 158) | G | A | oxa-13,19,28 | 22 |
| | | | | T | oxa-10,11,14, | |
| | | | | 16,17 | | |
| oxa | 449 oxa2 se | cttgtcgaaggaNcggcgttgc (SEQ ID NO: 159) | A | T | oxa-2,3 | 22 |
| (group | | | | C | oxa-15 | |
| 2) | oxa2 se | gctcctgcgccgagattgc (SEQ ID NO: 160) | | | oxa-2,15 | 19 |
| | oxa2 as | gcaatctcggcgcaggagc (SEQ ID NO: 161) | | | oxa-2,15 | 19 |
| | oxa2 3 se | ctgttcttgtgccgcgatagc (SEQ ID NO: 162) | | | oxa-3 | 21 |
| | oxa2 3 as | gctatcgcggcacaagaacag (SEQ ID NO: 163) | | | oxa-3 | 21 |

*se: sense probe; as: anti-sense probe; WT: wild-type
**N: Variable nucleotide in a capture probe for a SNP position, can be eiter A, T, G or C. On the array all four possible nucleotides are each represented with a capture probe. The indicated nucleotides are based on the capture probe sequence.
***N represents the respective nucleotide in the wild-type strain P. aeruginosa PAO1
****N represents the respective nucleotide for a SNIP position in the mutant sequence; Δ: deletion; ins: insertion
*****For SNPs, deletions or insertions either the respective amino acid change, frameshift, occurance of a premature stop codon or change of mutated gene compared to the parental gene is indicated.

What is claimed is:

1. A micro-array comprising a carrier and immobilized thereon in the form of a pattern nucleic acids comprising sequences specific for at least 8 determinants of Pseudomonas aeruginosa.

2. The micro-array according to claim 1, wherein said at least 8 determinants of Pseudomonas aeruginosa are selected from the group consisting of mexR, mexT, nfxB, mucA, parC, gyrA, exoU, exoS, exoT, pse, oxa, imp, vim, aac, aph and aad.

3. The micro-array according to claim 1, furher comprising controls.

4. The micro-array according to claim 3, wherein the controls are selected from the group consisting of gyrB, oprI, srv3.

5. The micro-array according to claim 1, wherein said carrier consists of glass, metal or plastics.

6. The micro-array according to claim 5, wherein said carrier consists of epoxy glass.

7. The micro-array according to claim 6, wherein said carrier is a microplate or a slide.

8. The micro-array according to claim 1, wherein the surface of said carrier comprises an area of at least 1 square centimeter.

9. The micro-array according to claim 1, wherein the nucleic acids are present on the carrier at a density of at least 100 molecules per square centimeter.

10. The micro-array according to claim 1, wherein said specific pattern allows the mapping of each nucleic acid to a specific position on said carrier and a specific analysis.

11. The micro-array according to claim 1, wherein said nucleic acids are immobilized via a spacer molecule.

12. A method for determining the presence of Pseudomonas aeruginosa in a sample comprising the steps of:
a) providing a micro-array comprising a carrier and immobilized thereon in the form of a pattern nucleic acids comprising sequences specific for at least 8 determinants of Pseudomonas aeruginosa,
b) contacting a sample to be analyzed with the micro-array under conditions allowing hybridization of complementary strands; and
c) determining, whether hybridisation occurrs.

13. The method according to claim 12, wherein said at least 8 determinants of Pseudomonas aeruginosa are randomly selected from the group consisting of mexR, mexT, nfxB, mucA, parC, gyrA, exoU, exoS, exoT, pse, oxa, imp, vim, aac, aph and aad.

14. The method according to claim 12, wherein the micro-array further comprises controls.

15. The method according to claim 12, wherein the controls are selected from the group consisting of gyrB, oprI, and srv3.

16. The method according to claim 12, wherein said sample contains nucleic acids comprising oligonucleotides and/or polynucleotides, having a length of about 10 to 100 nucleotides.

17. The method according to claim 16, wherein said oligonucleotides and/or polynucleotides are isolated from body tissues or fluids, suspected to contain Pseudomonas aeruginosa.

18. The method according to claim 16, wherein said oligonucleotides and/or polynucleotides are isolated from blood.

19. The method according to claim 16, wherein said nucleic acids are labelled with a marker molecule.

20. The method according to claim 19, wherein said marker molecule is selected from the group consisting of cyanine dyes, renaissance dyes, and fluorescent dyes.

21. The method of claim 20, wherein said cyanine dyes are selected from Cy3 and Cy5, said renaissance dyes are selected from ROX and E110, and said fluorescent dyes are selected from FAM and FITC.

22. A diagnostic kit for the detection of Pseudomonas aeruginosa infections, comprising nucleic acids specific for at least 8 determinants of Pseudomonas aeruginosa and/or a micro-array comprising a carrier and immobilized thereon in the form of a pattern nucleic acids comprising sequences specific for at least 8 determinants of Pseudomonas aeruginosa.

23. The kit according to claim 22, wherein the determinants are selected from the group consisting of mexR, mexT, nfxB, mucA, parC, gyrA, exoU, exoS, exoT, pse, oxa, imp, vim, aac, aph and aad.

24. The kit according to claim 22, further comprising controls.

25. The kit according to claim 22, further comprising buffers.


SEQUENCE LISTING (163 sequences)

SEQ ID NO: 1 - DNA, 31 bp, Artificial Sequence
  synthetic oligonucleotide
ttttttttttttttctagacagccactcata

SEQ ID NO: 2 - DNA, 32 bp, Artificial Sequence
  synthetic oligonucleotide
tttttttttttttgattggacgagtcaggagc

SEQ ID NO: 3 - DNA, 19 bp, Artificial Sequence
  synthetic oligonucleotide
gctcctgactcgtccaatc

SEQ ID NO: 4 - DNA, 31 bp, Artificial Sequence; identical to SEQ ID NO: 1
  synthetic oligonucleotide

SEQ ID NO: 5 - DNA, 17 bp, Artificial Sequence
  synthetic primer
gatgcccgcgctgatgg

SEQ ID NO: 6 - DNA, 20 bp, Artificial Sequence
  synthetic primer
aggcactggtcgaggagatg

SEQ ID NO: 7 - DNA, 22 bp, Artificial Sequence
  synthetic primer
atgcctgtcagtgatcctatgc

SEQ ID NO: 8 - DNA, 23 bp, Artificial Sequence
  synthetic primer
cgggtctcgaacggtgggtcctc

SEQ ID NO: 9 - DNA, 19 bp, Artificial Sequence
  synthetic primer
gcgacgctgaaggaactgg

SEQ ID NO: 10 - DNA, 18 bp, Artificial Sequence
  synthetic primer
ccgggcggtactggaata

SEQ ID NO: 11 - DNA, 19 bp, Artificial Sequence
  synthetic primer
cagcttgcggcgaggatgc

SEQ ID NO: 12 - DNA, 24 bp, Artificial Sequence
  synthetic primer
gtaccactgacggcggattgttgc

SEQ ID NO: 13 - DNA, 21 bp, Artificial Sequence
  synthetic primer
ctggatgccgattccaagcac

SEQ ID NO: 14 - DNA, 21 bp, Artificial Sequence
  synthetic primer
gaaggacttgggatcgtccgg

SEQ ID NO: 15 - DNA, 21 bp, Artificial Sequence
  synthetic primer
gacggcctgaagccggtgcac

SEQ ID NO: 16 - DNA, 21 bp, Artificial Sequence
  synthetic primer
gcccacggcgataccgctgga

SEQ ID NO: 17 - DNA, 20 bp, Artificial Sequence
  synthetic primer
ccgttgtggtgccgttgaag

SEQ ID NO: 18 - DNA, 20 bp, Artificial Sequence
  synthetic primer
ccagatgttcaccgactcgc

SEQ ID NO: 19 - DNA, 20 bp, Artificial Sequence
  synthetic primer
ccgttgtggtgccgttgaag

SEQ ID NO: 20 - DNA, 20 bp, Artificial Sequence
  synthetic primer
ccagatgttcaccgactcgc

SEQ ID NO: 21 - DNA, 20 bp, Artificial Sequence
  synthetic primer
gcgaggtcagcagagtatcg

SEQ ID NO: 22 - DNA, 20 bp, Artificial Sequence
  synthetic primer
ttcggcgtcactgtggatgc

SEQ ID NO: 23 - DNA, 22 bp, Artificial Sequence
  synthetic primer
aatcgccgtccaactgcatgcg

SEQ ID NO: 24 - DNA, 20 bp, Artificial Sequence
  synthetic primer
tgttcgccgaggtactgctc

SEQ ID NO: 25 - DNA, 22 bp, Artificial Sequence
  synthetic primer
cctgaccatccgtcgccacaac

SEQ ID NO: 26 - DNA, 20 bp, Artificial Sequence
  synthetic primer
cgcagcaggatgaagacgcc

SEQ ID NO: 27 - DNA, 18 bp, Artificial Sequence
  synthetic primer
gctctggctctggctgct

SEQ ID NO: 28 - DNA, 18 bp, Artificial Sequence
  synthetic primer
agggcacgctcgttagcc

SEQ ID NO: 29 - DNA, 19 bp, Artificial Sequence
  synthetic primer
cggnccagactcctacggg

SEQ ID NO: 30 - DNA, 18 bp, Artificial Sequence
  synthetic primer
ttaccgcggctgctggca

SEQ ID NO: 31 - DNA, 22 bp, Artificial Sequence
  synthetic primer
gctaaattactatgatgctgag

SEQ ID NO: 32 - DNA, 20 bp, Artificial Sequence
  synthetic primer
tattgccttaggagttgtcg

SEQ ID NO: 33 - DNA, 24 bp, Artificial Sequence
  synthetic primer
cagagaagttggcgaagtaagaat

SEQ ID NO: 34 - DNA, 19 bp, Artificial Sequence
  synthetic primer
aacccacccaacccaccat

SEQ ID NO: 35 - DNA, 20 bp, Artificial Sequence
  synthetic primer
gctcggcgctatttgaagaa

SEQ ID NO: 36 - DNA, 18 bp, Artificial Sequence
  synthetic primer
gcgcagcgtccgagttga

SEQ ID NO: 37 - DNA, 19 bp, Artificial Sequence
  synthetic primer
gacactccatttactgcta

SEQ ID NO: 38 - DNA, 24 bp, Artificial Sequence
  synthetic primer
attcagatgcatacgtggggatag

SEQ ID NO: 39 - DNA, 24 bp, Artificial Sequence
  synthetic primer
tgatacagcgtggggtgcgaaaaa

SEQ ID NO: 40 - DNA, 23 bp, Artificial Sequence
  synthetic primer
gtgccccggaatgacgaactgtg

SEQ ID NO: 41 - DNA, 22 bp, Artificial Sequence
  synthetic primer
ctcgaatgcctggcgtgtttga

SEQ ID NO: 42 - DNA, 21 bp, Artificial Sequence
  synthetic primer
gtggtggggcggagaagaagc

SEQ ID NO: 43 - DNA, 24 bp, Artificial Sequence
  synthetic primer
actggtctattcctcgcactcctg

SEQ ID NO: 44 - DNA, 22 bp, Artificial Sequence
  synthetic primer
cccccataactcttcgcctcat

SEQ ID NO: 45 - DNA, 17 bp, Artificial Sequence
  synthetic primer
gccggagactgcgagat

SEQ ID NO: 46 - DNA, 19 bp, Artificial Sequence
  synthetic primer
gcagtcgccctaaaacaaa

SEQ ID NO: 47 - DNA, 19 bp, Artificial Sequence
  synthetic primer
acgcttcaggtggctaatc

SEQ ID NO: 48 - DNA, 21 bp, Artificial Sequence
  synthetic primer
acaaagttaggtggctcaatg

SEQ ID NO: 49 - DNA, 18 bp, Artificial Sequence
  synthetic primer
ttcccccaaggcgtgacc

SEQ ID NO: 50 - DNA, 21 bp, Artificial Sequence
  synthetic primer
gcatacgcggaaggcaataac

SEQ ID NO: 51 - DNA, 24 bp, Artificial Sequence
  synthetic primer
gaagaactcgtccaatagcctgaa

SEQ ID NO: 52 - DNA, 21 bp, Artificial Sequence
  synthetic primer
gcgacgcctgcctgccaaatc

SEQ ID NO: 53 - DNA, 22 bp, Artificial Sequence
  synthetic primer
tatcagaggtagttggcgtcat

SEQ ID NO: 54 - DNA, 22 bp, Artificial Sequence
  synthetic primer
ttcaggaaccggatcaaagagt

SEQ ID NO: 55 - DNA, 21 bp, Artificial Sequence
  synthetic primer
tcaggaaccgggtcaaagaat

SEQ ID NO: 56 - DNA, 20 bp, Artificial Sequence
  synthetic primer
gagcgccatctggaatcaac

SEQ ID NO: 57 - DNA, 18 bp, Artificial Sequence
  synthetic primer
cggcacgcaagacctcaa

SEQ ID NO: 58 - DNA, 18 bp, Artificial Sequence
  synthetic primer
gcttggtgggcagacgaa

SEQ ID NO: 59 - DNA, 21 bp, Artificial Sequence
  synthetic capture probe
tcccaggtcccscaggttcag

SEQ ID NO: 60 - DNA, 20 bp, Artificial Sequence
  synthetic capture probe
tggcgtcccmggtcctgcag

SEQ ID NO: 61 - DNA, 20 bp, Artificial Sequence
  synthetic capture probe
ggatcttccrggtgatcagt

SEQ ID NO: 62 - DNA, 18 bp, Artificial Sequence
  synthetic capture probe
cgctggtccstggggttg

SEQ ID NO: 63 - DNA, 20 bp, Artificial Sequence
  synthetic capture probe
aggaagagckggaagctgcg

SEQ ID NO: 64 - DNA, 20 bp, Artificial Sequence
  synthetic capture probe
gcctccgcakgctggtggat

SEQ ID NO: 65 - DNA, 20 bp, Artificial Sequence
  synthetic capture probe
atgatggcmtccgcatgctg

SEQ ID NO: 66 - DNA, 19 bp, Artificial Sequence
  synthetic capture probe
cttgttccwccggggtgag

SEQ ID NO: 67 - DNA, 19 bp, Artificial Sequence
  synthetic capture probe
cagggtggcmtgttcctcc

SEQ ID NO: 68 - DNA, 20 bp, Artificial Sequence
  synthetic capture probe
ctggatgcgccggtccgcac

SEQ ID NO: 69 - DNA, 18 bp, Artificial Sequence
  synthetic capture probe
ctggatgcgcgtccgcac

SEQ ID NO: 70 - DNA, 20 bp, Artificial Sequence
  synthetic capture probe
cgctctggatggcgcgtccg

SEQ ID NO: 71 - DNA, 19 bp, Artificial Sequence
  synthetic capture probe
cgctctggatgcgcgtccg

SEQ ID NO: 72 - DNA, 20 bp, Artificial Sequence
  synthetic capture probe
caatcgagcatcgctctgga

SEQ ID NO: 73 - DNA, 19 bp, Artificial Sequence
  synthetic capture probe
caatcgagctcgctctgga

SEQ ID NO: 74 - DNA, 20 bp, Artificial Sequence
  synthetic capture probe
gcgtccgcacccgccatcag

SEQ ID NO: 75 - DNA, 20 bp, Artificial Sequence
  synthetic capture probe
tgctggaagaccgccatcag

SEQ ID NO: 76 - DNA, 20 bp, Artificial Sequence
  synthetic capture probe
ctggaagctggggttgcgct

SEQ ID NO: 77 - DNA, 19 bp, Artificial Sequence
  synthetic capture probe
gtcgctggggttgcgctcg

SEQ ID NO: 78 - DNA, 20 bp, Artificial Sequence
  synthetic capture probe
ggatggccagcccgtgagga

SEQ ID NO: 79 - DNA, 18 bp, Artificial Sequence
  synthetic capture probe
cagcccctcgtcggtgag

SEQ ID NO: 80 - DNA, 20 bp, Artificial Sequence
  synthetic capture probe
ggcctgttcccggggcaaac

SEQ ID NO: 81 - DNA, 18 bp, Artificial Sequence
  synthetic capture probe
ggggtgagcgggcaaaca

SEQ ID NO: 82 - DNA, 20 bp, Artificial Sequence
  synthetic capture probe
gcaccagggtttccaccggg

SEQ ID NO: 83 - DNA, 20 bp, Artificial Sequence
  synthetic capture probe
accagggtggcctgttccac

SEQ ID NO: 84 - DNA, 20 bp, Artificial Sequence
  synthetic capture probe
cgcgggtcasgctgcgttcg

SEQ ID NO: 85 - DNA, 20 bp, Artificial Sequence
  synthetic capture probe
acagtttctytgcggcgcgg

SEQ ID NO: 86 - DNA, 22 bp, Artificial Sequence
  synthetic capture probe
aggtcgccggmgaaggacacca

SEQ ID NO: 87 - DNA, 19 bp, Artificial Sequence
  synthetic capture probe
atcaatagaagttggcgcg

SEQ ID NO: 88 - DNA, 20 bp, Artificial Sequence
  synthetic capture probe
atcaatagatagttggcgcg

SEQ ID NO: 89 - DNA, 20 bp, Artificial Sequence
  synthetic capture probe
ccgcagaagckgtgcagggt

SEQ ID NO: 90 - DNA, 20 bp, Artificial Sequence
  synthetic capture probe
tgggtgaggkgttccttgat

SEQ ID NO: 91 - DNA, 21 bp, Artificial Sequence
  synthetic capture probe
ggccttgctctacgccggccg

SEQ ID NO: 92 - DNA, 20 bp, Artificial Sequence
  synthetic capture probe
ggccttgcttacgccggccg

SEQ ID NO: 93 - DNA, 18 bp, Artificial Sequence
  synthetic capture probe
ggtgcagggggccttgct

SEQ ID NO: 94 - DNA, 19 bp, Artificial Sequence
  synthetic capture probe
ggtgcagggtggccttgct

SEQ ID NO: 95 - DNA, 19 bp, Artificial Sequence
  synthetic capture probe
tgatctggttccagtacgg

SEQ ID NO: 96 - DNA, 18 bp, Artificial Sequence
  synthetic capture probe
tgatctggttcagtacgg

SEQ ID NO: 97 - DNA, 20 bp, Artificial Sequence
  synthetic capture probe
tccgggcggactggaatacc

SEQ ID NO: 98 - DNA, 21 bp, Artificial Sequence
  synthetic capture probe
tccgggcggtactggaatacc

SEQ ID NO: 99 - DNA, 20 bp, Artificial Sequence
  synthetic capture probe
tccgggcgggtactggaata

SEQ ID NO: 100 - DNA, 19 bp, Artificial Sequence
  synthetic capture probe
tccgggcggtactggaata

SEQ ID NO: 101 - DNA, 20 bp, Artificial Sequence
  synthetic capture probe
tagaccgcgrtgtcgccgtg

SEQ ID NO: 102 - DNA, 21 bp, Artificial Sequence
  synthetic capture probe
cacgatggtgkcgtagaccgc

SEQ ID NO: 103 - DNA, 20 bp, Artificial Sequence
  synthetic capture probe
tagcaggccragtcgccgtg

SEQ ID NO: 104 - DNA, 20 bp, Artificial Sequence
  synthetic capture probe
ccatggcctygtagcaggcc

SEQ ID NO: 105 - DNA, 18 bp, Artificial Sequence
  synthetic capture probe
cttcaccaggccatccgc

SEQ ID NO: 106 - DNA, 18 bp, Artificial Sequence
  synthetic capture probe
gcggatggcctggtgaag

SEQ ID NO: 107 - DNA, 17 bp, Artificial Sequence
  synthetic capture probe
gaaatcaccgcgctcgc

SEQ ID NO: 108 - DNA, 17 bp, Artificial Sequence
  synthetic capture probe
gcgagcgcggtgatttc

SEQ ID NO: 109 - DNA, 20 bp, Artificial Sequence
  synthetic capture probe
aagtgctccaccaggccatc

SEQ ID NO: 110 - DNA, 20 bp, Artificial Sequence
  synthetic capture probe
gatggcctggtggagcactt

SEQ ID NO: 111 - DNA, 19 bp, Artificial Sequence
  synthetic capture probe
cgtcttcggtagcggtcag

SEQ ID NO: 112 - DNA, 19 bp, Artificial Sequence
  synthetic capture probe
ctgaccgctaccgaagacg

SEQ ID NO: 113 - DNA, 24 bp, Artificial Sequence
  synthetic capture probe
ctgaagtggatgttgctgaaggtc

SEQ ID NO: 114 - DNA, 24 bp, Artificial Sequence
  synthetic capture probe
gaccttcagcaacatccacttcag

SEQ ID NO: 115 - DNA, 23 bp, Artificial Sequence
  synthetic capture probe
ttactgcccttcctcccaactta

SEQ ID NO: 116 - DNA, 23 bp, Artificial Sequence
  synthetic capture probe
taagttgggaggaagggcagtaa

SEQ ID NO: 117 - DNA, 21 bp, Artificial Sequence
  synthetic capture probe
gtcccctgttycgccatttgc

SEQ ID NO: 118 - DNA, 21 bp, Artificial Sequence
  synthetic capture probe
gtggtcccctyttgcgccatt

SEQ ID NO: 119 - DNA, 21 bp, Artificial Sequence
  synthetic capture probe
agggcgatcygcggggtggtc

SEQ ID NO: 120 - DNA, 21 bp, Artificial Sequence
  synthetic capture probe
gccccctgctyttcgctgtag

SEQ ID NO: 121 - DNA, 21 bp, Artificial Sequence
  synthetic capture probe
ggcgccccctyctcttcgctg

SEQ ID NO: 122 - DNA, 21 bp, Artificial Sequence
  synthetic capture probe
gtgatcacctycggcgccccc

SEQ ID NO: 123 - DNA, 21 bp, Artificial Sequence
  synthetic capture probe
agggtaggctccgcggtgcat

SEQ ID NO: 124 - DNA, 20 bp, Artificial Sequence
  synthetic capture probe
agggtaggctcgcggtgcat

SEQ ID NO: 125 - DNA, 20 bp, Artificial Sequence
  synthetic capture probe
atctgcggggggtcccctgt

SEQ ID NO: 126 - DNA, 21 bp, Artificial Sequence
  synthetic capture probe
atctgcggggtggtcccctgt

SEQ ID NO: 127 - DNA, 21 bp, Artificial Sequence
  synthetic capture probe
ccagcacggcacgggcctttc

SEQ ID NO: 128 - DNA, 20 bp, Artificial Sequence
  synthetic capture probe
ccagcacggccgggcctttc

SEQ ID NO: 129 - DNA, 20 bp, Artificial Sequence
  synthetic capture probe
acctgcggcgcccctgctct

SEQ ID NO: 130 - DNA, 21 bp, Artificial Sequence
  synthetic capture probe
acctgcggcgccccctgctct

SEQ ID NO: 131 - DNA, 21 bp, Artificial Sequence
  synthetic capture probe
cgggtatcgcatggacgagga

SEQ ID NO: 132 - DNA, 20 bp, Artificial Sequence
  synthetic capture probe
cgggtatcgctggacgagga

SEQ ID NO: 133 - DNA, 24 bp, Artificial Sequence
  synthetic capture probe
gaagaactcgtccaatagcctgaa

SEQ ID NO: 134 - DNA, 24 bp, Artificial Sequence
  synthetic capture probe
ttcaggctattggacgagttcttc

SEQ ID NO: 135 - DNA, 22 bp, Artificial Sequence
  synthetic capture probe
atgtgctttgtaggccagtcca

SEQ ID NO: 136 - DNA, 22 bp, Artificial Sequence
  synthetic capture probe
tggactggcctacaaagcacat

SEQ ID NO: 137 - DNA, 23 bp, Artificial Sequence
  synthetic capture probe
tttcatcaagccttacggtcacc

SEQ ID NO: 138 - DNA, 23 bp, Artificial Sequence
  synthetic capture probe
ggtgaccgtaaggcttgatgaaa

SEQ ID NO: 139 - DNA, 22 bp, Artificial Sequence
  synthetic capture probe
ggtgacttctatagcgcggagc

SEQ ID NO: 140 - DNA, 22 bp, Artificial Sequence
  synthetic capture probe
gctccgcgctatagaagtcacc

SEQ ID NO: 141 - DNA, 21 bp, Artificial Sequence
  synthetic capture probe
catacccaatcggctctccat

SEQ ID NO: 142 - DNA, 21 bp, Artificial Sequence
  synthetic capture probe
atggagagccgattgggtatg

SEQ ID NO: 143 - DNA, 17 bp, Artificial Sequence
  synthetic capture probe
aacgatgtgcggccggt

SEQ ID NO: 144 - DNA, 17 bp, Artificial Sequence
  synthetic capture probe
accggccgcacatcgtt

SEQ ID NO: 145 - DNA, 21 bp, Artificial Sequence
  synthetic capture probe
gttattgccttccgcgtatgc

SEQ ID NO: 146 - DNA, 21 bp, Artificial Sequence
  synthetic capture probe
gcatacgcggaaggcaataac

SEQ ID NO: 147 - DNA, 20 bp, Artificial Sequence
  synthetic capture probe
ctattgctgttccgcggtca

SEQ ID NO: 148 - DNA, 20 bp, Artificial Sequence
  synthetic capture probe
tgaccgcggaacagcaatag

SEQ ID NO: 149 - DNA, 24 bp, Artificial Sequence
  synthetic capture probe
aactcacgaccgaaaagatcaaga

SEQ ID NO: 150 - DNA, 24 bp, Artificial Sequence
  synthetic capture probe
tcttgatcttttcggtcgtgagtt

SEQ ID NO: 151 - DNA, 20 bp, Artificial Sequence
  synthetic capture probe
atcacttgthgtcatagttg

SEQ ID NO: 152 - DNA, 22 bp, Artificial Sequence
  synthetic capture probe
caaatcacchagcttaccttca

SEQ ID NO: 153 - DNA, 22 bp, Artificial Sequence
  synthetic capture probe
gagaattaagccactctattcc

SEQ ID NO: 154 - DNA, 22 bp, Artificial Sequence
  synthetic capture probe
ggaatagagtggcttaattctc

SEQ ID NO: 155 - DNA, 17 bp, Artificial Sequence
  synthetic capture probe
atcaacgccgccgacgc

SEQ ID NO: 156 - DNA, 17 bp, Artificial Sequence
  synthetic capture probe
gcgtcggcggcgttgat

SEQ ID NO: 157 - DNA, 21 bp, Artificial Sequence
  synthetic capture probe
gatattctgghtgccatagga

SEQ ID NO: 158 - DNA, 22 bp, Artificial Sequence
  synthetic capture probe
atttatttaaaratagagactc

SEQ ID NO: 159 - DNA, 22 bp, Artificial Sequence
  synthetic capture probe
cttgtcgaaggahcggcgttgc

SEQ ID NO: 160 - DNA, 19 bp, Artificial Sequence
  synthetic capture probe
gctcctgcgccgagattgc

SEQ ID NO: 161 - DNA, 19 bp, Artificial Sequence
  synthetic capture probe
gcaatctcggcgcaggagc

SEQ ID NO: 162 - DNA, 21 bp, Artificial Sequence
  synthetic capture probe
ctgttcttgtgccgcgatagc

SEQ ID NO: 163 - DNA, 21 bp, Artificial Sequence
  synthetic capture probe
gctatcgcggcacaagaacag